(19) United States
(12) Patent Application Publication
(10) Pub. No.: US 2016/0022720 A1
(43) Pub. Date: Jan. 28, 2016

(54) COMPOSITIONS AND METHODS FOR TREATING DISEASE STATES ASSOCIATED WITH ACTIVATED T CELLS AND/OR B CELLS

(71) Applicant: Children's Hospital Medical Center, Cincinnati, OH (US)

(72) Inventor: Michael Jordan, Wyoming, OH (US)

(21) Appl. No.: 14/449,798

(22) Filed: Aug. 1, 2014

Related U.S. Application Data
(60) Provisional application No. 61/861,556, filed on Aug. 2, 2013.

(51) Int. Cl. A61K 31/7048; A61K 31/4535; A61K 45/06; A61K 31/496

(52) U.S. Cl. CPC A61K 31/7048; A61K 31/496; A61K 31/4535; A61K 45/06

(57) ABSTRACT

Disclosed are combination therapies and related compositions that may contain one or more of a p53 potentiating agent, a DNA-damaging agent, an agent that inhibits cell cycle check point, and a pharmaceutically acceptable carrier. Also disclosed are methods of using such compositions for the treatment of conditions related to T cell and/or B cell activation in subjects in need of such treatment.

### CROSS-REFERENCE TO RELATED APPLICATIONS

This application claims the benefit of U.S. Application Ser. No. 61/861,556, filed Aug. 2, 2013, incorporated herein by reference in its entirety for all purposes.

### STATEMENT REGARDING FEDERALLY SPONSORED RESEARCH OR DEVELOPMENT

This invention was made with government support under HL091769 awarded by the National Institutes of Health. The government has certain rights in the invention.

### BACKGROUND

The inability to selectively target undesirable T cell and/or B cell responses driving a variety of immunopathological conditions including autoimmunity, allergy, inborn disorders of immune regulation, and allogeneic rejection is a fundamental clinical problem. Because of their central role in directing the immune response, T cells and B cells are a key component of nearly all immunopathological disorders: autoimmunity, allergy, immune regulatory disorders (such as HLH), allo-rejection, etc. These disorders have a combined multi-billion dollar effect on health care and are associated with substantial mortality and human suffering. Iatrogenic immune suppression (for treatment of autoimmunity or in the context of transplantation) is a major cause of infectious complications and deaths. New methods of immune modulation which avoid global suppression, but which efficiently and specifically target offending T cells could prevent this morbidity/mortality.

While progress has been made with newer immunosuppressive drugs, the underlying strategy remains one of global suppression in order to inhibit a few detrimental effector T cells and/or B cells. This broad inhibitory approach is the equivalent of declaring martial law on the immune system; curtailing the normal and beneficial actions of most adaptive immune cells in order to stop the rare rogue T cell or B cell. Current strategies have three major drawbacks: i) they lack immune specificity; ii) they increase the risks of opportunistic infections and cancers; and iii) they are associated with substantial agent specific organ toxicity. Thus, it is clear that there is a need to find novel and non-toxic means of controlling infrequent, yet injurious T and/or B cells, while maintaining beneficial memory and naïve T and/or B cells to combat pathogens. The instant disclosure addresses one or more of the aforementioned needs in the art.

### BRIEF SUMMARY

Disclosed herein are composition and methods useful for treatment of conditions or diseases caused or aggravated by increased T cell and/or B cell activity.

### BRIEF DESCRIPTION OF THE DRAWINGS

The drawings are for illustration purposes only, not for limitation.

FIG. 1 depicts inborn errors of immune regulation.

FIG. 2 depicts the effect of etoposide treatment on LCMV infected prf−/−mice having HLH-like disease. Prf−/−mice were treated with etoposide (ETOP) or drug carrier 5 days after LCMV-WE infection. LCMV-infected wild type mice treated with carrier are included for comparison. Mice were monitored for survival.

FIG. 3A depicts a live-gated dot-plot showing that etoposide selectively ablates activated effector T cells in LCMV-infected prf−/− and wild type mice. FIG. 3B depicts the CD8+ subpopulation. FIG. 3C depicts CD4+ subpopulation.

FIG. 4A shows that activated effector T cells were gehnerated in vitro by stimulation of transgenic T cells with peptide antigen for 2 days, followed by culture in IL-2 for 2 days, followed by either etoposide or drug carrier and assessed for apoptotic cell death by staining. FIG. 4B depicts survival of wild type (WT) or p53−/−cells that were infected with LCMV, cultured in IL-2 overnight, and cultured with varying concentrations of etoposide.

FIG. 5A depicts CD8+T cells stained directly ex vivo or after in vitro for serine 139 phosphorylation of histone H2A.X (gamma-H2AX). FIG. 5B depicts CD8+T cells stained directly ex vivo or after in vitro for serine 1981 phosphorylation of ATM. FIG. 5C depicts CD8+T cells stained directly ex vivo or after in vitro for serine 15 phosphorylation of p53. 5D depicts quantitative analysis of gh2ax staining in T cell populations in vivo or in vitro with or without etoposide treatment.

FIG. 6A depicts cell death after overnight culture by 7-AAD/PS staining. FIG. 6B depicts measurement of DNA damage by gamma-H2AX staining, in conjunction with cell cycle analysis, after a four hour exposure to etoposide. FIG. 6C depicts a graph of GammaH2A.X staining in response to increasing concentrations of etoposide. FIG. 6D depicts a graph showing topoisomerase 11β levels in isotype, resting, G1 activated, and S+G2/M activated T cells.

FIG. 7 depicts the pathways involved in apoptosis, cell cycle arrest and DNA repair, and the effect of chemo and radio-therapy.

FIG. 8A depicts activated T cell death following a titration of etoposide+/−5 uM nutlin. FIG. 8B depicts fold change of activated T cell numbers in vivo after varied treatments (in mice, low dose etoposide+/−mdm2 inhibitor, 5 days after LCMV infection, assessed on day 8. FIG. 8C depicts cell death in response to etoposide+/−a SIRT1 specific inhibitor or a MDM4 inhibitor.

FIG. 9A depicts cell death in response to etoposide+/−a RAD51 specific inhibitor or a CHK1/2 inhibitor. FIG. 9B depicts gammaH2.AX staining of activated T cells after overnight culture+/−AZD7762. FIG. 9C depicts fold change of activated T cell numbers in vivo after varied treatments (in mice, low dose etoposide+/−chk1/2 inhibitor, 5 days after LCMV infection, assessed on day 8).

FIG. 10 depicts the effects of p53 potentiators and DDR inhibitors on activated T cells in vivo.

FIG. 11 depicts the effects of etoposide and p53 potentiators on reactivated memory cells in vivo.

FIG. 12A shows that inhibitors of Chk1/2 or Wee1 synergize with etoposide for killing of activated, but not resting T cells. FIG. 12B shows gammaH2AX staining of activated T cells after overnight culture+/−a titration of AZC7762, analyzed by cell cycle status. FIG. 12C depicts LCMV-infected animals treated with low dose etoposide (10 mg/kg)+/−AZD7762 (25 mg/kg) on day 5 of infection and assessment of antigen specific T cells as assessed on day 8 by MHC tetramer staining.

FIG. 13 depicts the clinical score over time in a hemophagocytic lymphohistiocytosis (HLH) model in response to varying treatment, including etoposide, nutlin, AZD7762, AZD7762+nutlin, and carrier.

FIG. 14 depicts the clinical score over time in an experimental autoimmune encephalomyelitis model in response to etoposide and MK-1775 + Nutlin.

### DETAILED DESCRIPTION

Definitions

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “individual,” “host,” “subject,” and “patient” are used interchangeably to refer to an animal that is the object of treatment, observation and/or experiment. “Animal” includes vertebrates and invertebrates, such as fish, shellfish, reptiles, birds, and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. Pharmaceutically acceptable carriers include a wide range of known diluents (i.e., solvents), fillers, extending agents, binders, suspending agents, disintegrates, surfactants, lubricants, excipients, wetting agents and the like commonly used in this field. These carriers may be used singly or in combination according to the form of the pharmaceutical preparation, and may further encompass “pharmaceutically acceptable excipients” as defined herein.

As used herein, “pharmaceutically acceptable excipient” means any other component added to a pharmaceutical formulation other than the active ingredient and which is capable of bulking-up formulations that contain potent active ingredients (thus often referred to as “bulking agents,” “fillers,” or “diluents”) to allow convenient and accurate dispensation of a drug substance when producing a dosage form. Excipients may be added to facilitate manufacture, enhance stability, control release, enhance product characteristics, enhance bioavailability drug absorption or solubility, or other pharmacokinetic considerations, enhance patient acceptability, etc. Pharmaceutical excipients include, for example, carriers, fillers, binders, disintegrants, lubricants, glidants, colors, preservatives, suspending agents, dispersing agents, film formers, buffer agents, pH adjusters, preservatives etc. The selection of appropriate excipients also depends upon the route of administration and the dosage form, as well as the active ingredient and other factors, and will be readily understood by one of ordinary skill in the art.

As used herein, the term “therapeutically effective amount” means the total amount of each active component of the pharmaceutical composition or method that is sufficient to show a meaningful patient benefit, e.g., healing of chronic conditions or in an increase in rate of healing of such conditions, or in a reduction in aberrant conditions. This includes both therapeutic and prophylactic treatments. Accordingly, the compounds can be used at very early stages of a disease, or before early onset, or after significant progression. When applied to an individual active ingredient, administered alone, the term refers to that ingredient alone. When applied to a combination, the term refers to combined amounts of the active ingredients that result in the therapeutic effect, whether administered in combination, serially or simultaneously.

Other features, objects, and advantages of the present invention will be apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

As described herein, Applicant has surprisingly discovered synergistic mechanisms between potential cancer therapeutics and therapies useful for immunopathological disorders. Without being limited by theory, it is believed that modulation of p53 and the DDR (DNA Damage Response) may provide non-genotoxic methods to manipulate the DNA damage response for immunomodulatory therapies. In particular, Applicant has found that activated T cells display a strong spontaneous DDR in vivo, that manipulation of the DDR response and p53 activity can promote selective elimination of activated T cells, and that DNA damaging agents such as etoposide may be a therapeutic for immunopathological conditions such as HLH based on the ability of such drugs to selectively ablate activated T cells. (FIG. 1 depicts inborn errors of immune regulation.) T cells and B cells undergo analogous selection processes in the thymus or marrow respectively. They have similar life cycles; they enter the periphery as quiescent naïve cells. Once they encounter antigen, via analogous receptors (T cell receptor/B cell receptor) they undergo a burst of proliferation. The antigen responding population swells massively, then contracts in an analogous fashion for both T and B cells. These similarities of life cycle suggest that the compositions and methods described herein that target only activated (not quiescent) T cells would target activated B cells equally as well. Indeed, DNA damaging drugs such as cyclophosphamide are thought to treat disorders such as Lupus erythematosus primarily by their ability to kill B cells. As T cells transition between their developmental states—naive, activated, effector, quiescent memory, and activated memory—they exhibit unique attributes that may be exploited to kill such activated cells. Acutely-activated T cells display a strong DDR in vivo. Etoposide, a chemotherapeutic agent in wide clinical use, ablates activated T cells while sparing naïve and quiescent memory T cells. Compounds that enhance p53-mediated signaling, such as MDM2 inhibitors (which release p53 to act as a cellular executioner), or inhibitors of cell cycle check point, have been discovered to greatly potentiate etoposide-ablation of activated T cells. Thus, the intrinsic DNA damage response of activated T cells pushes them to the threshold of death, and augmenting p53 activity pushes them beyond this threshold into apoptosis. It is believed that B cells respond similarly. Because quiescent naïve and memory T cells and B cells do not display a significant DDR and do not have activated p53, they are resistant to treatments that increase p53 signaling strength. These insights suggest strategies for the development of novel and highly selective forms immune suppression which are minimally or non-genotoxic and better tolerated than current approaches. Studying the impact of the DNA damage response (DDR) on the survival of mature T cells is a novel area of study. Specifically, the hypothesis that DDR modulation may provide non-genotoxic immunomodulatory therapies is innovative. Further, the concept of the use of potential cancer therapeutics as therapeutic agents for immunopathologic disorders is believed to be a novel and an unexplored concept.

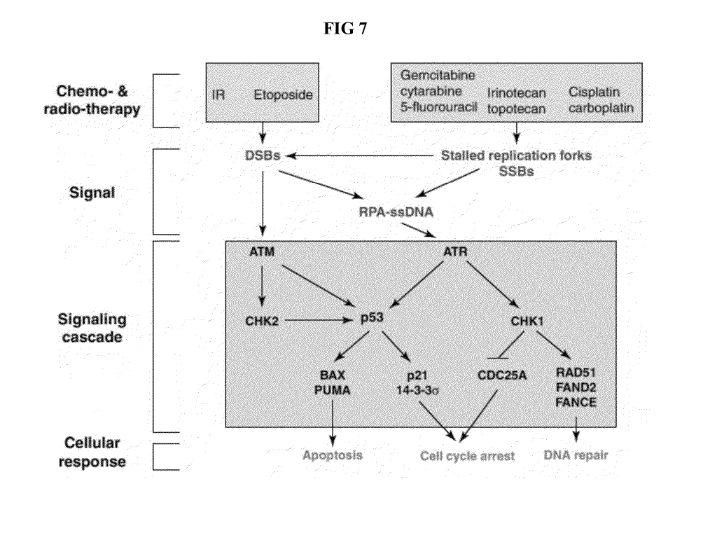

DNA-damaging chemotherapeutic agents have an important, if limited, clinical role as immunosuppressive agents (e.g. treatment of lupus nephritis, multiple sclerosis, rheumatoid arthritis, and for prevention of graft-versus-host disease). DNA-damaging agents (etoposide, cyclophosphamide, methotrexate, etc.) are used in various settings to control deleterious auto- or allo-immune responses. FIG. 7 depicts the pathways involved in apoptosis, cell cycle arrest and DNA repair, and the effect of chemo and radio-therapy. However, off-target toxicity, such as myelo-suppression may be significant. Their application, however, has been largely empirical (with minimal mechanistic insight) and has been limited by adverse effects such as bone marrow suppression. Development of non-genotoxic DNA damage response (DDR) modulators and deeper understanding of the mechanisms by which these agents kill immune cells would allow newer approaches with less off-target toxicity.

Pharmacologic manipulation of the DDR and p53-mediated responses is an active area of investigation in experimental and translational cancer therapeutics. Applicant has uncovered unappreciated immunologic effects of these strategies which suggest additional and novel therapeutic potential. This cross fertilization between disparate fields (DNA repair, oncology, and immunology) is likely to drive innovative studies with significant potential to shift current therapeutic paradigms and improve human health.

Applicant has studied the effects of etoposide on T cells, an agent that is the backbone of therapy (via a previously unknown mechanism of action) for the fatal immunoregulatory disorder hemophagocytic lymphohistiocytosis (HLH). The fundamental importance of multiple immune regulatory pathways to human health is demonstrated by inborn immune regulatory disorders, such as HLH. The study of rare disorders gives unique insights into human immune function. HLH, in particular, is a valuable disorder to study because it is a clear example of a purely T cell driven immunopathologic disorder.

HLH is characterized by excessive T cell activation and is a prototype for T cell-driven immunopathologic disorders. It is caused by mutations in perforin (and related genes). While treatment with etoposide is effective for many patients, HLH is a notoriously difficult disorder to treat. Though etoposide-based therapy has increased long-term survival from approximately 0% to 55%, patients with HLH continue to die due to resistant disease or therapy-related toxicity.

Applicant has further developed a robust model of this disorder, defining the key role of T cells in its pathogenesis and now defining the mechanisms of action for current therapies. A now widely used model of HLH involving lymphocytic choriomeningitis virus (LCMV) infection of perforin-deficient mice has been developed by Applicant. Using this model, Applicant has found that etoposide is capable of rescuing animals from disease development by depleting activated T cells with remarkable selectivity. Applicant further has found that etoposide appears to engage multiple apoptotic pathways that may provide additional therapeutic targets, and that etoposide acts synergistically with several classes of agents to selectively and effectively ablate activated T cells. Moreover, etoposide is believed to have similar effects on activated T cells in wild type animals due to experimental evidence that etoposide is therapeutic in experimental autoimmune encephalitis, suggesting a broader utility for this drug.

Mechanistically, without intended to be limited by theory, it is Applicant's belief that antigenic activation of T cells and/or B cells renders them uniquely susceptible to p53-mediated apoptosis, which may be therapeutically triggered with agents that augment p53-signaling, while affording survival of naïve and pre-existing memory T and/or B cells.

In one aspect, by using the disclosed combinations of active agents as described herein, Applicant has found that unwanted T cells and/or B cells may be acutely activated in vivo and selectively targeted for apoptotic elimination using activators of p53, avoiding broad and blunt suppression of T cells and/or B cells which may lead to undesirable side effects. For example, by using the disclosed novel combination of active agents as described herein, beneficial immunity may be spared while undesirable T cells and/or B cells are purged with minimal toxicity in a broad array of clinical contexts, thus allowing for targeted treatment of T cell and/or B cell associated pathological conditions with improved efficacy and decreased toxicity and/or side effects.

The instant disclosure further provides methods and compositions effective for a variety of disease states, and embody, in some aspects, therapies that are antigen specific (selective for recently activated T cells) but for which the antigen is not necessarily defined (unlike conventional antigen specific approaches. The instant disclosure is based, in part, on the novel observations that etoposide is therapeutic for HLDH based on its ability to selectively ablate activated T cells, activated T cells display a strong spontaneous DDR in vivo, and ‘synthetic’ manipulation of the DDR/p53 can promote selective elimination of activated T cells and etoposide is therapeutic for HLH based on its ability to selectively ablate activated T cells.

Because activated T cells are accumulating DNA damage at a substantial rate, if one briefly (or episodically) inhibited DNA repair, it is believed that one could selectively kill activated lymphocytes. The two pathways for repair of double-stranded DNA breaks are homologous recombination (HR) and non-homologous end joining (NHEJ). Applicant has found that inhibition of HR (eg, by inhibition of Rad51, using Ri-1), but not inhibition of NHEJ (eg—testing a variety of DNA-PK inhibitors) led to substantial synergy with etoposide in vitro (data not shown).

Because activated T cells appear to be accumulating DNA damage as a consequence of rapid cell cycling, it was reasoned that inhibiting molecules which enforce cell cycle checkpoints would lead (indirectly) to further accumulation of DNA damage, activation of p53, and apoptosis. Applicant found that a Chk1/2 inhibitor (AZD7762) and a Wee1 inhibitor (MK-1775) both selectively kill activated T cells and potentiate etoposide killing in vitro (FIG. 7a). Culturing activated T cells in AZD7762 led to increasing DNA damage (gammaH2AX). This damage accumulated mostly in cells which were in S phase or G2/M, suggesting that repair of damage sustained during DNA replication was inhibited (FIG. 7b). Similar to MDM2 inhibition, inhibition of Chk1/2 synergized potently with etoposide in vivo for the selective depletion of activated T cells.

Compositions

Net, Applicant has discovered that inhibition of cell cycle checkpoints as well as potentiation of p53 is capable of pushing activated T cells over the brink to apoptosis. In particular, a p53 potentiating agent such as an inhibitor of MDM2, and checkpoint inhibitors such as inhibitors of CHK1/2 or Wee1 act synergistically without requiring DNA damaging agents like etoposide, to deplete harmful T cells. Further, Applicant has discovered synergy between chemotherapeutic agents and the combination of a p53 potentiating agent (such as MDM2 inhibitors) and/or checkpoint inhibition such that the DNA damage response can be manipulated for immunotherapy. ATR inhibitors may also be used for inhibition of the DNA repair mechanism in combination with any of the above agents and/or in combination with an MDM2 inhibitor or etoposide.

In one aspect, compositions that may comprise an agent selected from a p53 potentiating agent; a DNA-damaging agent, DNA repair inhibitor/cell cycle checkpoint inhibitor, and combinations thereof; and a pharmaceutically acceptable carrier are disclosed. As used herein, the phrase “DNA repair inhibitor/cell cycle checkpoint inhibitor” is used to include agents that inhibit the activity of cellular signaling agents involved in DNA repair and/or which are involved in controlling the cell cycle checkpoint mechanism that ensures the fidelity of cell division in eukaryotic cells.

In one aspect, the compositions and methods may employ the combination of an inhibitor of MDM2 and an inhibitor of CHK1/2, an inhibitor of Wee 1, or an inhibitor of ATR for treatment of disease states as disclosed herein, particularly such disease states involving activated T-cells. Applicant has found impressive synergy in vitro and in vivo of these agents without use of an exogenous non-specific DNA damaging agent such as etoposide.

In one aspect, the composition may comprise a p53 potentiating agent; a DNA damaging agent; a DNA repair inhibitor/cell cycle checkpoint inhibitor; and a pharmaceutically acceptable carrier.

In one aspect, the composition may comprise a p53 potentiating agent; a DNA-damaging agent; and a pharmaceutically acceptable carrier.

In one aspect, the composition may comprise a p53 potentiating agent; a DNA repair inhibitor/cell checkpoint inhibitor and a pharmaceutically acceptable carrier.

In one aspect, the composition may comprise a DNA damaging agent; a DNA repair inhibitor; and a pharmaceutically acceptable carrier.

In one aspect, the composition may comprise a DNA repair inhibitor/cell cycle checkpoint inhibitor, and a pharmaceutically acceptable carrier.

In some aspects, the compositions may be formulated as a single oral dosage form.

p53 Potentiating Agents

P53 is widely considered to be a master integrator of cellular stresses, promoting cell cycle arrest, senescence, DNA repair, and apoptosis in varying measures based on diverse inputs and contexts. MDM2 (along with MDM4) is a major regulator of p53 activity, sequestering and ubiquinating it. Rationally designed small molecule inhibitors of MDM2 have been developed, which “release” p53. MDM2 inhibitors (the prototypical drug, called nutlin-3, referred to as “nutlin” herein) are currently in clinical trials for the treatment of cancers. Because nutlin enhances p53 function, it may also protect non-malignant cells (with non-mutant p53) from accumulating DNA damage in response to chemotherapy. The DDR promotes DNA repair and survival by a variety of mechanisms, including cell cycle arrest. Concurrent with cell cycle arrest, repair mechanisms are engaged.

In one aspect, the p53 potentiating agent may be selected from an MDM2 inhibitor, an MDM4 inhibitor, a dual MDM2/MDM4 inhibitor, a SIRT 1 inhibitors, and a combination thereof In one aspect, the p53 potentiating agent may comprise a MDM2 inhibitor. In one aspect, the p53 potentiating agent may comprise a nutlin compound, such as nutlin 1, nutlin 2, nutlin 3, or combinations thereof In one aspect, the p53 potentiating agent may comprise nutlin 3.

MDM2/MDM4 Inhibitors

P53 potentiating agents may include, for example, for example, MDM2 (also known as HDMX) and/or MDM4 (also known as MDMX) inhibitors. Examples of which include, for example, analogs of cys-imidazolie (nutlin 1, nutlin 2, nutlin 3), spiro-oxindole, benzodiazepinedione, terphynyl, quilinol, chalcone, and sulfonamide. In other aspects, the p53 potentiating agent may include

In one aspect, the p53 potentiating agent may be RG7388 (RO5503781), available from ChemiTek, Indianapolis, Ind., having the following structure:

In one aspect, the p53 potentiating agent may be AMG-232 (AMG232), described in Sun et al, “Discovery of AMG 232, a Potent, Selective, and Orally Bioavailable MDM2-p53 Inhibitor in Clinical Development” Journal of Med. Chem., 2013, having the following structure:

In one aspect, the p53 potentiating agent is RO5045337, having the following structure:

RO5045337 is believed to bind to MDM2, thereby preventing the binding of the MDM2 protein to the transcriptional activation domain of the tumor suppressor protein p53. By preventing this MDM2-p53 interaction, the proteosome-mediated enzymatic degradation of p53 is inhibited and the transcriptional activity of p53 is restored, which may result in the restoration of p53 signaling and thus the p53-mediated induction of tumor cell apoptosis.

In one aspect, the p53 potentiating agent may be CGM097, (available from Novartis). CGM097 is an orally bioavailable HDM2 (human homolog of double minute 2) antagonist with potential antineoplastic activity. Upon oral administration, p53/HDM2 interaction inhibitor CGM097 inhibits the binding of the HDM2 protein to the transcriptional activation domain of the tumor suppressor protein p53. By preventing this HDM2-p53 interaction, the proteosome-mediated enzymatic degradation of p53 is inhibited, which may result in the restoration of p53 signaling and, thus, the p53-mediated induction of tumor cell apoptosis.

In one aspect, the p53 potentiating agent may be RG7112, a small-molecule MDM2 antagonist (See, e.g., Tovar et al., “MDM2 Small-Molecule Antagonist RG7112 Activates p53 Signaling and Regresses Human Tumors in Preclinical Cancer Models,” Cancer Res; 73(8) (2013)) having the following structure:

In one aspect, the p53 potentiating agent may be a Nutlin, a cis-imidazoline analog that inhibits the interaction between mdm2 and tumor suppressor p53. In one aspect, the p53 potentiating agent may be Nutlin-3a (Structure shown in Table 2).

In one aspect, the p53 potentiating agent may be MI-219, having the following structure:

Additional MDM2 and MDM4 inhibitors that may be suitable for use in the methods and compositions herein are listed in the following Table 1 (Wade et al, “MDM2, MDMX and p53 in oncogenesis and cancer therapy,” Nature Reviews, Vol 13 (2013)) and Table 2 (Vassilev, “MDM2 inhibitors for cancer therapy,” Trends in Mol. Med., Vol 13, No. 1 (2006)). Other MDM2 and/or MDM4 inhibitors known or identified in the art may further be useful in the described compositions and methods, including, but not limited to, those described in Zhao et al., “Small Molecule Inhibitors of MDM2-p53 and MDMX-p53 Interactions as New Cancer Therapeutics”, BioDiscovery 2013; 8: 4; DOI: 10.7750/BioDiscovery.2013.8.4.

TABLE 1
Targeting approach, Compound and Class of Agents that Target MDM2 and MDM4

| Targeting | | | | Proposed working |
|---|---|---|---|---|
| approach | Compound | Class | Target | mechanism |
| Modulating | NSC207895 | Small | MDMX | Inhibits MDMX |
| protein expression | (REF. 88) | molecule | | transcription |
| | 17-AAG | Small | HSP90 | HSP90 inhibitor |
| | | molecule | | |
| Targeting protein- | Nutlin 3a | Small | MDM2 N-terminal p53-binding | Disrupts p53-MDM2 |
| protein interaction | | molecule | pocket | interaction |
| | MI-219 | Small | MDM2 N-terminal p53-binding | Disrupts p53-MDM2 |
| | (REF. 97) | molecule | pocket | interaction |
| | SJ-172550 | Small | MDMX N-terminal p53-binding | Disrupts p53-MDMX |
| | (REF. 105) | molecule | pocket | interaction |
| | RO-5963 | Small | Both MDM2 and MDMX | Disrupts p53-MDM2 and |
| | (REF. 103) | molecule | N-terminal p53-binding pocket | p53-MDMX interactions |
| | WK 298 | Small | MDMX | Disrupts p53-MDMX |
| | (REF. 173) | molecule | | interaction |
| | AM-8553 | Small | MDM2 N-terminal p53-binding | Disrupts p53-MDM2 |
| | (REF. 174) | molecule | pocket | interaction |
| | SAH-p53-8 | Peptidic | Both MDM2 and MDMX | Disrupts p53-MDM2 and |
| | (REF. 110) | compound | N-terminal p53-binding pocket | p53-MDMX interactions |
| | PMI peptide¹¹² | Peptidic | Both MDM2 and MDMX | Disrupts p53-MDM2 and |
| | | compound | N-terminal p53-binding pocket | p53-MDMX interactions |
| | pDI peptide¹¹¹ | Peptide | Both MDM2 and MDMX | Disrupts p53-MDM2 and |
| | | | N-terminal p53-binding pocket | p53-MDMX interactions |
| | RITA¹⁵³ | Small | p53 N-terminal domain | Disrupts p53-MDM2 |
| | | molecule | | interaction |
| Targeting E3 | HLI98 | Small | MDM2 | Inhibits MDM2 ubiquitin |
| ubiquitin ligase | (REF. 115) | molecule | | ligase activity |
| activity | MPD¹¹⁶ | Small | MDM2 RING domain | Inhibits MDM2 ubiquitin |
| | | molecule | | ligase activity |
| | MEL23 and MEL24 | Small | MDM2 | Inhibits MDM2 ubiquitin |
| | (REF. 117) | molecule | | ligase activity |
| Activating p53 via | JNJ-26854165 | Small | MDM2 | Inhibits p53-MDM2- |
| other mechanisms | | molecule | | proteasome interaction |

indicates data missing or illegible when filed

```
TABLE 2
Small molecule MDM2 inhibitors
  HL198C
  Nutlin-3
  Benzodiazepines
  RITA
  Spiro-oxindoles
  Quilinois
```

SIRT Inhibitors

Sirtuins, or class III histone deacetylases (HDACs) are a group of NAD+ dependent enzymes with protein deacetylase and/or ADP-ribosyl transferase activity. Mammals express seven sirtuin homologs. Sirtuins directly affect multiple substrates including tumor suppressors such as p53. As such, in some aspects, the p53 potentiating agent may comprise a sirtuin (SIRT) inhibitor, such as a SIRT-1 inhibitor, a SIRT-2 inhibitor, or combinations thereof Non-limiting examples of p53 potentiating agents include sirtinol, salermide, EX-527, splitomycin, cambinol, suramin, tenovins (including tenovin-1 and/or tenovin-6), 3,2′,3′,4′-tetrahydroxychalcone, or combinations thereof (See Table 3.) Other SIRT inhibitors known or identified in the art may further be useful in the described compositions and methods.

TABLE 3
Examples of SIRT Inhibitors

| | |
|---|---|
| sirtinol | |
| salermide | |
| EX-527 | |
| splitomycin | |
| cambinol | |
| suramin | |
| tenovin-1 | |
| tenovin-6 | |

DNA-Damaging Agents

In one aspect, the compositions and methods may employ one or more DNA-damaging agents as contemplated herein. In one aspect, the DNA damaging agent may be selected from a topoisomerase type I inhibitor, a topoisomerase type II inhibitor, an alkylating agent, an antimetabolite, a cytotoxic antibiotic, a purine analogue, a dihydrofolate reductase inhibitor, and combinations thereof.

DNA damaging agents for use with the described compositions and methods described herein may include, for example, topoisomerase type I inhibitors (e.g., Irinotecan, Topotecan, Camptothecin, lamellarin D); topoisomerase type II inhibitors (e.g., etoposide (VP-16), etoposide phosphate, teniposide, doxorubicin, daunorubicin, mitoxantrone, amsacrine, ellipticines, aurintricarboxylic acid, HU-331 (a quinolone synthesized from cannabidiol), fluroquinolones (such as ciprofloxacin), ICRF-193, genistein); alkylating agents (e.g., Cisplatin, Carboplatin, Oxaliplatin, cyclophosphamide); antimetabolites (e.g., methotrexate); cytotoxic antibiotics (e.g., Acitinomycin, anthracyclines (doxorubicin, daunorubicin, valrubicin, idarubicin, epirubicin), Bleomycin, plicamycin, mitomycin); Purine analogues (e.g., purines such as azathioprine, mercaptopurine and pyrimidines such as thioguanine, fludarabine, pentostatin, cladribine); and dihydrofolate reductase inhibitors

In one aspect, the DNA-damaging agent may comprise a topoisomerase type II inhibitor such as, for example, etoposide, teniposide, doxorubicin, daunorubicin, mitoxantrone, amsacrine, ellipticines, aurintricarboxylic acid, HU-331, and combinations thereof In one aspect, the DNA-damaging agent may comprise etoposide. Other DNA-damaging agents known or identified in the art may further be useful in the described compositions and methods.

DNA Repair Inhibitors/Cell Cycle Checkpoint Inhibitors

In one aspect, the compositions and methods may employ one or more agents that inhibit DNA repair, including cell cycle checkpoint inhibitors, as described herein. Cell cycle checkpoint inhibitors indirectly inhibit timely DNA repair. In one aspect, the DNA repair inhibitor/cell cycle checkpoint inhibitor may be selected from a CHK1/2 Inhibitor, a Rad51 Inhibitor, a Wee1 inhibitor, an ATR inhibitor and combinations thereof.

DNA repair inhibitors may include, for example, a CHK 1/2 inhibitor, such as one or more listed in Table 4. In one aspect, the CHK1CHK2 inhibitor may be urea based AZD7762.

DNA repair inhibitors may further be a cell cycle checkpoint inhibitor, for example, an inhibitor of Wee1. Without intending to be limited by theory, it is believed that by inhibiting molecules that enforce cell cycle checkpoints, this would indirectly lead to further accumulation of DNA damage, activation of p53 and apoptosis. Applicant found that a Chk1/2 inhibitor (AZD7762) and a Wee1 inhibitor (MK-1775, structure shown below) both selectively kill activated T cells and potentiate etoposide killing of activated T cells in vitro. Culturing activated T cells in AZD7762 led to increasing DNA damage, which accumulated mostly in cells that were in S phase or G2/M, suggesting that repair or damage sustained during DNA replication was inhibited. Similar to MDM2 inhibition, inhibition of Chk1/2 synergized potently with etoposide in vivo for the selective depletion of activated T cells.

Wee1 inhibitors that may be used in the instant compositions and methods include, for example, 4-(2-Chlorophenyl)-9-hydroxypyrrolo[3,4-c]carbazole-1,3-(2H,6H)-dione (C₂₀H₁₁ClN₂O₃); 6-Butyl-4-(2-chlorophenyl)-9-hydroxypyrrolo[3,4-c]carbazole-1,3-(2H,6H)-dione (C₂₄H₁₉ClN₂O₃); 4-(2-Phenyl)-9-hydroxypyrrolo[3,4-c]carbazole-1,3-(2H,6H)-dione (C₂₀H₁₂N₂O₃.H₂O); and 6-(2,6-Dichlorophenyl)-2-(4-(2-(diethylaminoethoxy)-phenylamino)-8-methyl-8H-pyrido[2,3-d]pyrimidin-7-one (C₂₆H₂₇Cl₂N₅O₂.2HCl), all available from Merck Millipore, and MK-1775 (Selleck Chemicals, Houston, Tex.), having the following structure:

Pharmaceutically acceptable salts of the aforementioned compounds are also within the scope of the invention and will be readily understood by one of ordinary skill in the art.

TABLE 4
CHK1 inhibitors

| | |
|---|---|
| UCN-01 | |
| AZD7762 | |
| PF477736 | |
| SCH900776 | |

UCN-01 is available from Sigma-Aldrich; AZD7762 is available from Cayman Chemical; PF477736 is available from Selleckchem.com; and SCH900776 is available from Selleckchem.com.

In other aspects, the DNA repair inhibitor/cell cycle checkpoint inhibitor of the disclosed compositions may comprise a Rad inhibitor. In one aspect, the RAD inhibitor may be, for example, a Rad 51 inhibitor such as RI-1, or RI-2. Overexpression of RAD51 is believed to be common in cancer cells and represent a potential therapeutic target in oncology. (Budke, et al., J. of Med. Chem, 2012). A chemical inhibitor of RAD51, RI-1 has the following formula: 3-chloro-1-(3,4-dichlorophenyl)-4-morpholino-1H-pyrrole-2,5-dione.

In another aspect, the RAD inhibitor may comprise a RAD51 inhibitor having the chemical formula: 1-(3,4-dichlorophenyl)-3-(4-metholyphenyl)-4-morpholino-1H-pyrrole-2,5,-dione. (“RI-2”)

In other aspects, the DNA repair inhibitor/cell cycle checkpoint inhibitor of the disclosed compositions may comprise an inhibitor of ATR Inhibitors of ATR are known in the art, and include, for example, AZ20, VE-821, ETP-46464, VE-822, BEZ235, Torin 2, CGK 733, and Wortmannin, all of which are available from Selleckchem.com. The structures of these compounds are shown in the following Table.

| ATR Inhibitor Name | ATR Inhibitor Structure |
|---|---|
| AZ20 | |
| VE-821 | |
| ETP-46464 | |
| VE-822 | |
| BEZ235 | |
| Torin 2 | |
| CGK733 | |
| Wortmannin | |

Composition Forms

The compositions described herein may take a variety of forms, depending on the desired route of administration to an individual. For example, the compositions may be formulated as liquid compositions, such as for use as an intravenous formulation, or oral liquid formulations. In other aspects, the compositions may be formulated as solid compositions, such as in the form of a tablet, a capsule, or the like, suitable for administration to an individual in need thereof. Further, the compositions may be formulated in any suitable carrier and include any excipients as are well known and used in the art.

Methods

In one aspect, a method of treating a condition caused or aggravated by activated T cells and/or B cells, comprising the step of administering a composition as described herein, is disclosed. In one aspect, the condition may be an immunological condition. In one aspect, the condition may be an immunological condition selected from allergies, autoimmune conditions, allo-immune conditions, and other pathological immune reactivities. The condition may be selected from hemophagocytic lympohohistiocytosis, graft versus host disease, EAE, lupus nephritis, multiple sclerosis, rheumatoid arthritis, autoimmune encephalitis, allogenic graft rejection, transfusion reactions, allergies, anti-drug immune responses, and/or blood product reactions. In one aspect, the condition may be hemophagocytic lympohohistiocytosis (HLH).

In one aspect, the composition may be administered via a bolus injection or via continuous infusion to an individual in need thereof. In another aspect, the composition may be administered orally via a single oral dosage form, or using a combination of dosage forms.

Non-limiting examples of suitable pharmaceutically acceptable diluents and carriers include phosphate buffered saline solutions, water, emulsions including oil/water emulsions, various types of wetting agents such as detergents, and sterile solutions. Compositions comprising such carriers can be formulated by well known conventional methods. Compositions can also comprise liquid or viscous compositions that can coat and/or line the surface of the GI tract, thereby placing the active compounds in direct proximity with the epithelial cells.

Compounds, or mixtures of compounds described herein, can be formulated into pharmaceutical composition comprising a pharmaceutically acceptable carrier and other excipients as apparent to the skilled worker. Such composition can additionally contain effective amounts of other compounds, especially for the treatment of conditions, diseases, and/or disorders described herein.

Some embodiments comprise the administration of a pharmaceutically effective quantity of active agent or its pharmaceutically acceptable salts or esters, active agent analogs or their pharmaceutically acceptable salts or esters, or a combination thereof.

The compositions and preparations may contain at least 0.1% of active agent. The percentage of the compositions and preparations can, of course, be varied, and can contain between about 2% and 60% of the weight of the amount administered. The percentage of the compositions and preparations may contain between about 2, 5, 10, or 15% and 30, 35, 40, 45, 50, 55, or 60% of the weight of the amount administered. The amount of active compounds in such pharmaceutically useful compositions and preparations is such that a suitable dosage will be obtained.

The disclosed active agents may form salts. Reference to a compound of the active agent herein is understood to include reference to salts thereof, unless otherwise indicated. The term “salt(s)”, as employed herein, denotes acidic and/or basic salts formed with inorganic and/or organic acids and bases. In addition, when an active agent contains both a basic moiety, such as, but not limited to an amine or a pyridine or imidazole ring, and an acidic moiety, such as, but not limited to a carboxylic acid, zwitterions (“inner salts”) can be formed and are included within the term “salt(s)” as used herein. Pharmaceutically acceptable (e.g., non-toxic, physiologically acceptable) salts are preferred, although other salts are also useful, e.g., in isolation or purification steps, which can be employed during preparation. Salts of the compounds of the active agent can be formed, for example, by reacting a compound of the active agent with an amount of acid or base, such as an equivalent amount, in a medium such as one in which the salt precipitates or in an aqueous medium followed by lyophilization.

Pharmaceutically acceptable salts include, but are not limited to, pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like.

The compounds can be formulated in various forms, including solid and liquid forms, such as tablets, gel, syrup, powder, aerosol, etc.

The compositions may contain physiologically acceptable diluents, fillers, lubricants, excipients, solvents, binders, stabilizers, and the like. Diluents that can be used in the compositions include but are not limited to dicalcium phosphate, calcium sulphate, lactose, cellulose, kaolin, mannitol, sodium chloride, dry starch, powdered sugar and for prolonged release tablet-hydroxy propyl methyl cellulose (HPMC). The binders that can be used in the compositions include but are not limited to starch, gelatin and fillers such as sucrose, glucose, dextrose and lactose.

Natural and synthetic gums that can be used in the compositions include but are not limited to sodium alginate, ghatti gum, carboxymethyl cellulose, methyl cellulose, polyvinyl pyrrolidone and veegum. Excipients that can be used in the compositions include but are not limited to microcrystalline cellulose, calcium sulfate, dicalcium phosphate, starch, magnesium stearate, lactose, and sucrose. Stabilizers that can be used include but are not limited to polysaccharides such as acacia, agar, alginic acid, guar gum and tragacanth, amphotsics such as gelatin and synthetic and semi-synthetic polymers such as carbomer resins, cellulose ethers and carboxymethyl chitin.

Solvents that can be used include but are not limited to Ringers solution, water, distilled water, dimethyl sulfoxide to 50% in water, propylene glycol (neat or in water), phosphate buffered saline, balanced salt solution, glycol and other conventional fluids.

The dosages and dosage regimen in which the compounds are administered will vary according to the dosage form, mode of administration, the condition being treated and particulars of the patient being treated. Accordingly, optimal therapeutic concentrations will be best determined at the time and place through routine experimentation.

The compounds may also be used enterally. Orally, the compounds may be administered at the rate of 100 μg to 100 mg per day per kg of body weight. Orally, the compounds may be suitably administered at the rate of about 100, 150, 200, 250, 300, 350, 400, 450, or 500 μg to about 1, 5, 10, 25, 50, 75, 100 mg per day per kg of body weight. The required dose can be administered in one or more portions. For oral administration, suitable forms are, for example, tablets, gel, aerosols, pills, dragees, syrups, suspensions, emulsions, solutions, powders and granules; one method of administration includes using a suitable form containing from 1 mg to about 500 mg of active substance. In one aspect, administration may comprise using a suitable form containing from about 1, 2, 5, 10, 25, or 50 mg to about 100, 200, 300, 400, 500 mg of active substance.

The compounds may also be administered parenterally in the form of solutions or suspensions for intravenous or intramuscular perfusions or injections. In that case, the compounds may be administered at the rate of about 10 μg to 10 mg per day per kg of body weight; one method of administration may consist of using solutions or suspensions containing approximately from 0.01 mg to 1 mg of active substance per ml. The compounds may be administered at the rate of about 10, 20, 30, 40, 50, 60, 70, 80, 90, or 100 μg to 1, 2, 3, 4, 5, 6, 7, 8, 9, or 10 mg per day per kg of body weight; in one aspect, solutions or suspensions containing approximately from 0.01, 0.02, 0.03, 0.04, or 0.5 mg to 0.1, 0.2, 0.3, 0.4, 0.5, 0.6, 0.7, 0.8, 0.9, or 1 mg of active substance per ml may be used.

The compounds can be used in a substantially similar manner to other known anti-cancer agents for treating (both chemopreventively and therapeutically) various cancers. For the anti-cancer dose to be administered, whether a single dose, multiple dose, or a daily dose, will of course vary with the particular compound employed because of the varying potency of the compound, the chosen route of administration, the size of the recipient, the type of cancer, and the nature of the patient's condition. The dosage to be administered is not subject to definite bounds, but it will usually be an effective amount, or the equivalent on a molar basis of the pharmacologically active free form produced from a dosage formulation upon the metabolic release of the active drug to achieve its desired pharmacological and physiological effects. For example, an oncologist skilled in the art of cancer treatment will be able to ascertain, without undue experimentation, appropriate protocols for the effective administration of the compounds related to cancer therapy, such as by referring to the earlier published studies on compounds found to have anti-cancer properties.

The active compounds and/or pharmaceutical compositions of the embodiments disclosed herein can be administered according to various routes, such as by injection, for example local or systemic injection(s). Intratumoral injections maybe used for treating existing cancers. Other administration routes can be used as well, such as intramuscular, intravenous, intradermic, subcutaneous, etc. Furthermore, repeated injections can be performed, if needed, although it is believed that limited injections will be needed in view of the efficacy of the compounds.

For ex vivo administration, the active agent can be administered by any standard method that would maintain viability of the cells, such as by adding it to culture medium (appropriate for the target cells) and adding this medium directly to the cells. As is known in the art, any medium used in this method can be aqueous and non-toxic so as not to render the cells non-viable. In addition, it can contain standard nutrients for maintaining viability of cells, if desired. For in vivo administration, the complex can be added to, for example, to a pharmaceutically acceptable carrier, e.g., saline and buffered saline, and administered by any of several means known in the art. Examples of administration include parenteral administration, e.g., by intravenous injection including regional perfusion through a blood vessel supplying the tissues(s) or organ(s) having the target cell(s), or by inhalation of an aerosol, subcutaneous or intramuscular injection, topical administration such as to skin wounds and lesions, direct transfection into, e.g., bone marrow cells prepared for transplantation and subsequent transplantation into the subject, and direct transfection into an organ that is subsequently transplanted into the subject. Further administration methods include oral administration, particularly when the active agent is encapsulated, or rectal administration, particularly when the active agent is in suppository form.

It is contemplated that such target cells can be located within a subject or human patient, in which case a safe and effective amount of the active agent, in pharmacologically acceptable form, would be administered to the patient. Generally speaking, it is contemplated that useful pharmaceutical compositions may include the selected active compound derivative in a convenient amount, e.g., from about 0.001% to about 10% (w/w) that is diluted in a pharmacologically or physiologically acceptable carrier, such as, for example, phosphate buffered saline. The route of administration and ultimate amount of material that is administered to the subject under such circumstances will depend upon the intended application and will be apparent to those of skill in the art in light of the examples which follow.

Any composition chosen should be of low or non-toxicity to the cell. Toxicity for any given compound can vary with the concentration of compound used. It is also beneficial if the compound chosen is metabolized or eliminated by the body and if this metabolism or elimination is done in a manner that will not be harmfully toxic.

The compound may be administered such that a therapeutically effective concentration of the compound is in contact with the affected cells of the body. The dose administered to a subject, particularly a human, may be sufficient to effect a therapeutic response in the subject over a reasonable period of time. The dose may be determined by the strength of the particular compound employed and the condition of the subject, as well as the body weight of the subject to be treated. The existence, nature, and extent of any adverse side effects that might accompany the administration of a particular compound also will determine the size of the dose and the particular route of administration employed with a particular patient. In general, the compounds may be therapeutically effective at low doses. The generally useful dose range may be from about 0.001 mM, or less, to about 100 mM, or more. The effective dose range may be from about 0.01, 0.05, 0.1, 0.5, 0.6, 0.7, 0.8, or 0.9 mM, to about 1, 2, 3, 4, 5, 6, 7, 8, 9, or 10 mM. Accordingly, the compounds may be generally administered in low doses.

The pharmaceutical composition may further comprise a pharmaceutically acceptable carrier. The resulting preparation may incorporate, if necessary, one or more solubilizing agent, buffers, preservatives, colorants, perfumes, flavorings and the like that are widely used in the field of pharmaceutical preparation.

The proportion of the active ingredient to be contained in the disclosed compositions may be determined by one of ordinary skill in the art using art recognized methods.

The disclosed compounds may be formulated into a dosage form selected from the group consisting of tablets, capsules, granules, pills, injections, solutions, emulsions, suspensions, and syrups. The form and administration route for the pharmaceutical composition are not limited and can be suitably selected. For example, tablets, capsules, granules, pills, syrups, solutions, emulsions, and suspensions may be administered orally. Additionally, injections (e.g. subcutaneous, intravenous, intramuscular, and intraperitoneal) may be administered intravenously either singly or in combination with a conventional replenisher containing glucose, amino acid and/or the like, or may be singly administered intramuscularly, intracutaneously, subcutaneously and/or intraperitoneally.

The disclosed compositions may be prepared according to a method known in the pharmaceutical field of this kind using a pharmaceutically acceptable carrier. For example, oral forms such as tablets, capsules, granules, pills and the like are prepared according to known methods using excipients such as saccharose, lactose, glucose, starch, mannitol and the like; binders such as syrup, gum arabic, sorbitol, tragacanth, methylcellulose, polyvinylpyrrolidone and the like; disintegrates such as starch, carboxymethylcellulose or the calcium salt thereof, microcrystalline cellulose, polyethylene glycol and the like; lubricants such as talc, magnesium stearate, calcium stearate, silica and the like; and wetting agents such as sodium laurate, glycerol and the like.

Injections, solutions, emulsions, suspensions, syrups and the like may be prepared according to a known method suitably using solvents for dissolving the active ingredient, such as ethyl alcohol, isopropyl alcohol, propylene glycol, 1,3-butylene glycol, polyethylene glycol, sesame oil and the like; surfactants such as sorbitan fatty acid ester, polyoxyethylenesorbitan fatty acid ester, polyoxyethylene fatty acid ester, polyoxyethylene of hydrogenated castor oil, lecithin and the like; suspending agents such as cellulose derivatives including carboxymethylcellulose sodium, methylcellulose and the like, natural gums including tragacanth, gum arabic and the like; and preservatives such as parahydroxybenzoic acid esters, benzalkonium chloride, sorbic acid salts and the like.

The compounds can be administered orally, topically, parenterally, by inhalation or spray, vaginally, rectally or sublingually in dosage unit formulations. The term “administration by injection” includes but is not limited to: intravenous, intraarticular, intramuscular, subcutaneous and parenteral injections, as well as use of infusion techniques. Dermal administration can include topical application or transdermal administration. One or more compounds can be present in association with one or more non-toxic pharmaceutically acceptable carriers and if desired other active ingredients.

Compositions intended for oral use can be prepared according to any suitable method known to the art for the manufacture of pharmaceutical compositions. Such compositions can contain one or more agents selected from the group consisting of diluents, sweetening agents, flavoring agents, coloring agents and preserving agents in order to provide palatable preparations. Tablets contain the active ingredient in admixture with non-toxic pharmaceutically acceptable excipients that are suitable for the manufacture of tablets. These excipients can be, for example, inert diluents, such as calcium carbonate, sodium carbonate, lactose, calcium phosphate or sodium phosphate; granulating and disintegrating agents, for example, corn starch, or alginic acid; and binding agents, for example magnesium stearate, stearic acid or talc. The tablets can be uncoated or they can be coated by known techniques to delay disintegration and adsorption in the gastrointestinal tract and thereby provide a sustained action over a longer period. For example, a time delay material such as glyceryl monostearate or glyceryl distearate can be employed. These compounds can also be prepared in solid, rapidly released form.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions containing the active materials in admixture with excipients suitable for the manufacture of aqueous suspensions can also be used. Such excipients are suspending agents, for example sodium carboxymethylcellulose, methylcellulose, hydroxypropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethylene oxycetanol, or condensation products of ethylene oxide with partial esters derived from fatty acids and hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, can also be present.

The compounds can also be in the form of non-aqueous liquid formulations, e.g., oily suspensions which can be formulated by suspending the active ingredients in a vegetable oil, for example arachis oil, olive oil, sesame oil or peanut oil, or in a mineral oil such as liquid paraffin. The oily suspensions can contain a thickening agent, for example beeswax, hard paraffin or cetyl alcohol. Sweetening agents such as those set forth above, and flavoring agents can be added to provide palatable oral preparations. These compositions can be preserved by the addition of an anti-oxidant such as ascorbic acid.

Compounds may also be administrated transdermally using methods known to those skilled in the art. For example, a solution or suspension of an active agent in a suitable volatile solvent optionally containing penetration enhancing agents can be combined with additional additives known to those skilled in the art, such as matrix materials and bacteriocides. After sterilization, the resulting mixture can be formulated following known procedures into dosage forms. In addition, on treatment with emulsifying agents and water, a solution or suspension of an active agent can be formulated into a lotion or salve.

Suitable solvents for processing transdermal delivery systems are known to those skilled in the art, and include lower alcohols such as ethanol or isopropyl alcohol, lower ketones such as acetone, lower carboxylic acid esters such as ethyl acetate, polar ethers such as tetrahydrofuran, lower hydrocarbons such as hexane, cyclohexane or benzene, or halogenated hydrocarbons such as dichloromethane, chloroform, trichlorotrifluoroethane, or trichlorofluoroethane. Suitable solvents can also include mixtures of one or more materials selected from lower alcohols, lower ketones, lower carboxylic acid esters, polar ethers, lower hydrocarbons, halogenated hydrocarbons.

Suitable penetration enhancing materials for transdermal delivery system are known to those skilled in the art, and include, for example, monohydroxy or polyhydroxy alcohols such as ethanol, propylene glycol or benzyl alcohol, saturated or unsaturated C8-C18 fatty alcohols such as lauryl alcohol or cetyl alcohol, saturated or unsaturated C8-C18 fatty acids such as stearic acid, saturated or unsaturated fatty esters with up to 24 carbons such as methyl, ethyl, propyl, isopropyl, n-butyl, sec-butyl, isobutyl, tertbutyl or monoglycerin esters of acetic acid, capronic acid, lauric acid, myristinic acid, stearic acid, or palmitic acid, or diesters of saturated or unsaturated dicarboxylic acids with a total of up to about 24 carbons such as diisopropyl adipate, diisobutyl adipate, diisopropyl sebacate, diisopropyl maleate, or diisopropyl fumarate. Additional penetration enhancing materials include phosphatidyl derivatives such as lecithin or cephalin, terpenes, amides, ketones, ureas and their derivatives, and ethers such as dimethyl isosorbid and diethyleneglycol monoethyl ether. Suitable penetration enhancing formulations can also include mixtures of one or more materials selected from monohydroxy or polyhydroxy alcohols, saturated or unsaturated C8-C18 fatty alcohols, saturated or unsaturated C8-C18 fatty acids, saturated or unsaturated fatty esters with up to 24 carbons, diesters of saturated or unsaturated discarboxylic acids with a total of up to 24 carbons, phosphatidyl derivatives, terpenes, amides, ketones, ureas and their derivatives, and ethers.

Suitable binding materials for transdermal delivery systems are known to those skilled in the art and include polyacrylates, silicones, polyurethanes, block polymers, styrenebutadiene copolymers, and natural and synthetic rubbers. Cellulose ethers, derivatized polyethylenes, and silicates can also be used as matrix components. Additional additives, such as viscous resins or oils can be added to increase the viscosity of the matrix.

Pharmaceutical compositions may also be in the form of oil-in-water emulsions. The oil phase can be a vegetable oil, for example olive oil or arachis oil, or a mineral oil, for example, liquid paraffin or mixtures of these. Suitable emulsifying agents can be naturally-occurring gums, for example, gum acacia or gum tragacanth, naturally-occurring phosphatides, for example, soy bean, lecithin, and esters or partial esters derived from fatty acids and hexitol anhydrides, for example, sorbitan monooleate, and condensation products of the said partial esters with ethylene oxide, for example, polyoxyethylene sorbitan monooleate. The emulsions can also contain sweetening and flavoring agents. Syrups and elixirs can be formulated with sweetening agents, for example glycerol, propylene glycol, sorbitol or sucrose. Such formulations can also contain a demulcent, a preservative and flavoring and coloring agents.

The compounds can also be administered in the form of suppositories for rectal or vaginal administration of the drug. These compositions can be prepared by mixing the drug with a suitable nonirritating excipient which is solid at ordinary temperatures but liquid at the rectal temperature or vaginal temperature and will therefore melt in the rectum or vagina to release the drug. Such materials include cocoa butter and polyethylene glycols.

It will be appreciated by those skilled in the art that the particular method of administration will depend on a variety of factors, all of which are considered routinely when administering therapeutics. It will also be understood, however, that the specific dose level for any given patient will depend upon a variety of factors, including, the activity of the specific compound employed, the age of the patient, the body weight of the patient, the general health of the patient, the gender of the patient, the diet of the patient, time of administration, route of administration, rate of excretion, drug combinations, and the severity of the condition undergoing therapy. It will be further appreciated by one skilled in the art that the optimal course of treatment, i.e., the mode of treatment and the daily number of doses of an active agent or a pharmaceutically acceptable salt thereof given for a defined number of days, can be ascertained by those skilled in the art using conventional treatment tests.

Also disclosed are methods of reducing the number of activated T cells and/or B cells in vivo, comprising the step of administering a composition as disclosed herein.

In another aspect, a method of selectively modulating immune function is disclosed, comprising administering a composition as described herein, wherein the selective modulation avoids global immune suppression.

In a further aspect, a method of inducing selective tolerance to an agent activating an immune response of an individual, is disclosed, comprising the step of administering a composition as described herein to an individual in need thereof.

In a yet further aspect, a method of enhancing the effectiveness of etoposide is disclosed, comprising the step of administering an agent selected from a p53 potentiating agent, a DNA repair inhibitor, or a combination thereof.

### EXAMPLES

Defects of perforin (and functionally related genes) cause HLH, a fatal immune regulatory disorder characterized by excessive T cell activation due to defective feedback to APCs, often triggered by infection. Applicant has demonstrated that HLH can be modeled in LCMV-infected prf−/−mice, recreating all disease features and demonstrating the critical role that T cells and T cell-derived cytokines play in driving disease progression. Etoposide, a topoisomerase II inhibiting chemotherapeutic agent in wide use for treatment of cancer, was discovered to be therapeutic for HLH over 30 years ago. Subsequent international studies have established etoposide as the standard of care for HLH, though no mechanism of action was ever defined. Applicant has found that etoposide is highly therapeutic in murine HLH, at does which are equivalent to those used in HLH patients. It allowed survival, decreased inflammatory cytokines/disease-specific inflammatory markers, and alleviated pancytopenia that develops in these mice. It has been found by Applicant that etoposide exerts these therapeutic effects via selective destruction of acutely-activated CD8+ and CD4+ T cells and suppression of inflammatory cytokines. This depletion was remarkably potent (nearly 100 fold depletion of activated cells and specific (quiescent naïve and memory T cells were largely spared).

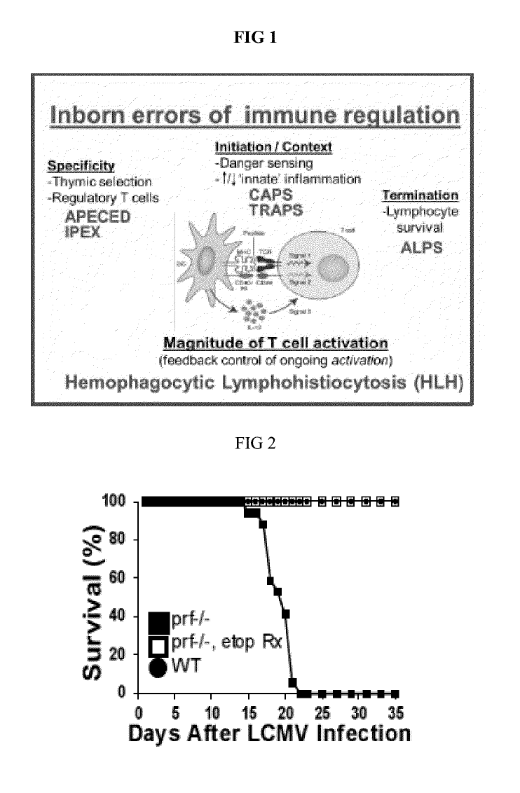

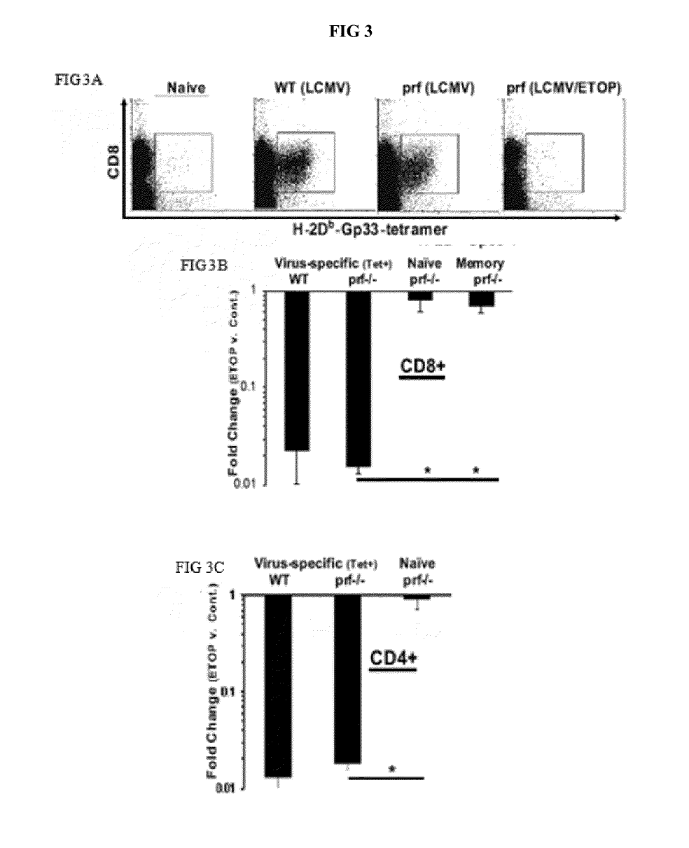

FIG. 2 shows that etoposide treatment rescues LCMV-infected prf−/− mice from HLH-like disease. Prf−/− mice were treated with etoposide (ETOP), or drug carrier 5 days after LCMV-WE infection. LCMV-infected wild type mice treated with carrier are included for comparison. Mice were monitored for survival. FIG. 3 shows that etoposide selectively ablates activated effector T cells in LCMV-infected prf−/− and WT mice. LCMV-WE infected prf−/− and WT mice were treated with etoposide (ETOP) or carrier 5 days post-infection. Eight days after infection, LCMV specific T cells were enumerated using MHC multimeric staining reagents (Db-GP33 and IAb-GP61). Representative live-gated dot-plots are shown in (A) and CD8+ subpopulations are quantitated in (B); CD4+ subpopulations are shown in (C). Fold change of T cell populations after etoposide was calculated by dividing the absolute number of each population by the size of that population in carrier treated, LCMV infected mice of the same genotype (n>15 for each). To conservatively account for the limits of detection with tetramer staining, animals in which NO antigen-specific T cells could be found were scored as ‘100 fold depletion’ (approx. ⅓ of mice were in this category). Naïve cells are defined as CD441o. Quiescent memory CD8+ T cells were generated in vivo by transfer of Ova-specific T cells (OT1), followed by priming with vaccinia-ova, followed by an interval of >1 month prior to LCMV challenge. OT1 T cells were enumerated by congenic markers. *p<0.01

Moreover, Applicant has found that etoposide acts in an essentially identical fashion in LCMV-infected wild type (WT) mice, suggesting that its immunomodulatory qualities are not restricted to the context of HLH. Following up on this observation, Applicant has found with collaborators that etoposide is highly therapeutic in experimental autoimmune encephalitis a widely studied model for human multiple sclerosis.

Etoposide causes double stranded DNA breaks via inhibition of topoisomerase II. DNA damage triggers a well studied series of events, including activation of ATM/ATR, p53 and downstream mechanisms leading to DNA repair, senescence/cell cycle arrest, and.or cell death. P53 mediates etoposide-triggered apoptotic death of thymocytes and many malignant cell types. However, p53 is not clearly implicated in etoposide driven dealth of mature, activated T cells in vitro.

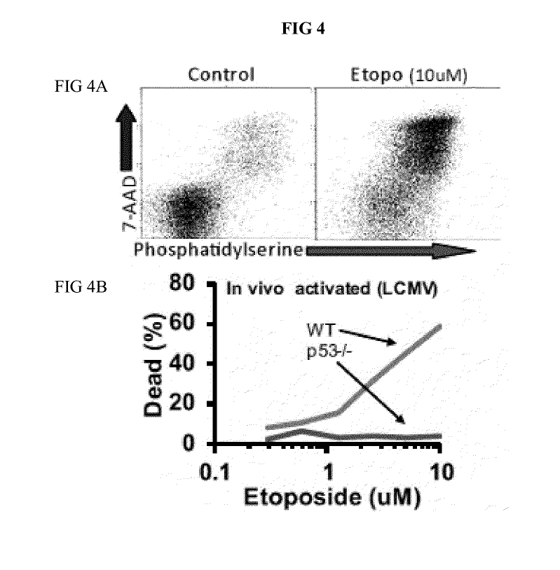

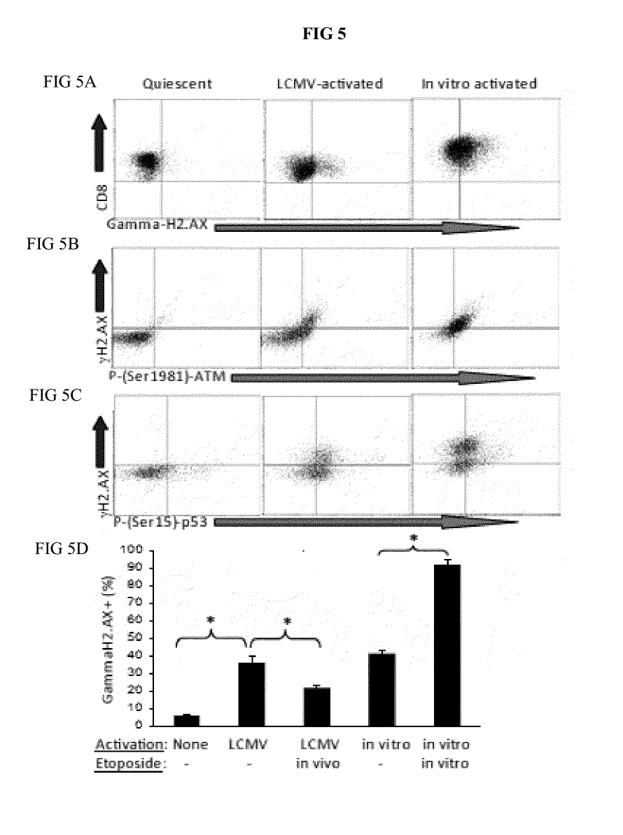

Etoposide triggered apoptotic death of activated T cells is also largely p53-dependent (FIG. 4). FIG. 4 shows that etoposide kills activated T cells via a p53-dependent mechanism. Referring to FIG. 4A, activated effector T cells were generated in vitro by stimulation of transgenic T cells (P14) with peptide antigen for 2 days, followed by culture in IL-2 for 2 days. They were then treated for 14 hours with either etoposide or drug carrier and assessed for apoptotic cell death by staining with 7-AAD and A647-labeled MFG-E8 protein (a superior, fixable PS stain, see Asano, K., et al., Masking of phosphatidylserine inhibits apoptotic cell engulfment and induces autoantibody production in mice. J Exp Med, 2004. 200(4): p. 459-67.). Referring to FIG. 4B, wild type (WT) or p53−/− mice were infected with LCMV. Six days later, spleen cells were removed and cultured in IL-2 overnight. Live cells were purified with ficoll gradient centrifugation and then cultured for 14 hours with varying concentrations of etoposide. Death was assessed by 7-AAD/PS staining.

Notably, Applicant found that activated (but not resting) T cells display a strong DNA damage response (DDR) signature (both in vivo and in vitro) as measured by several markers, without prior exposure to etoposide. In vitro etoposide treatment led to increased measures of DNA damage while in vivo treatment led to decreased numbers of T cells with measurable DNA damage (FIG. 5). This decrease suggests a threshold effect-activated T cells are selectively lost, leaving quiescent T cells with lower amounts of DNA damage. Applicant has measured gamma-H2AX (the phospohorylation of serine 1398 on histone H2AX) the most sensitive and widely used marker of double stranded DNA breaks, along with multiple other markers of DDR activation.

After exposure to etoposide, Applicant found that activated T cells display a substantial increase in DNA damage, downstream DDR signaling, and apoptosis induction, compared to their baseline and to resting T cells. Thus, activated T cells have both increased spontaneous DNA damage, and heightened activation of the DDR after exposure to exogenous genotoxins. Without intending to be limited by theory, it is believed that that there are at least two potential reasons why activated T cells display an increased DDR: damage due to “replication stress” and to increased metabolic stresses, such as reactive oxygen species. All cells display some evidence of damage to DNA when dividing, however, lymphocytes undergo uniquely intense and extremely rapid cell division after antigenic activation. Thus, while metabolic stresses probably also contribute, it is reasoned by Applicant that replication stress is likely to be the major contributor to increased baseline damage. Furthermore, the process of DNA replication including risky unwinding of DNA using topoisomerases, is likely to explain increased DNA damage with exposure to exogenous genotoxic agents. Activated murine and human T cells having varying cell division rates were challenged with etoposide. As division rates slowed, baseline DNA damage and sensitivity to etoposide decreased markedly, suggesting that cell cycling rate relates to etoposide effectiveness. These data suggest that activated T cells survive at the edge of a DNA damage ‘death precipice’ due to the rapid cell division they experience after cognate antigenic exposure. When they are subjected to additional DDR-activating stresses or agents (such as etoposide) they are readily pushed over into apoptosis because of their uniquely precarious situation.

Activated T cells display a strong spontaneous DDR in vivo, and ‘synthetic’ manipulation of the DDR/p53 can promote selective elimination of activated T cells. When exposed to additional p53-activating stresses or agents (such as etoposide), they are readily pushed over into an apoptotic abyss. FIG. 5 shows that activated T cells display a spontaneous DDR in vivo and in vitro, without exposure to DNA-damaging drugs. Referring to FIG. 5 A-C, CD8+ T cells were stained directly ex vivo (uninfected or day 6 LCMV-infected prf−/− mice), or after in vitro stimulated (P14 T cells as in FIG. 3) for serine 139 phosphorylation of histone H2A.X (referred to as gamma-H2.AX), serine 1981 phosphorylation of ATM, and serine 15 phosphorylation of p53, in CD8+ T cells from uninfected mice, day 6 LCMV infected mice, or transgenic T cells (P14) antigenically stimulated in vitro. Referring to FIG. 5D, the percentage of CD8+ T cells which were gamma-H2.AX+ are quantitated from either D-6 LCMV-infected mice which were treated with etoposide (50 mg/kg ip, on day 5) or activated P14 T cells, cultured for 4 hours with 5 uM etoposide. *p<0.01 n.b: GammaH2Ax stain in panels B and C are performed post fixation, which decreases stain sensitivity.

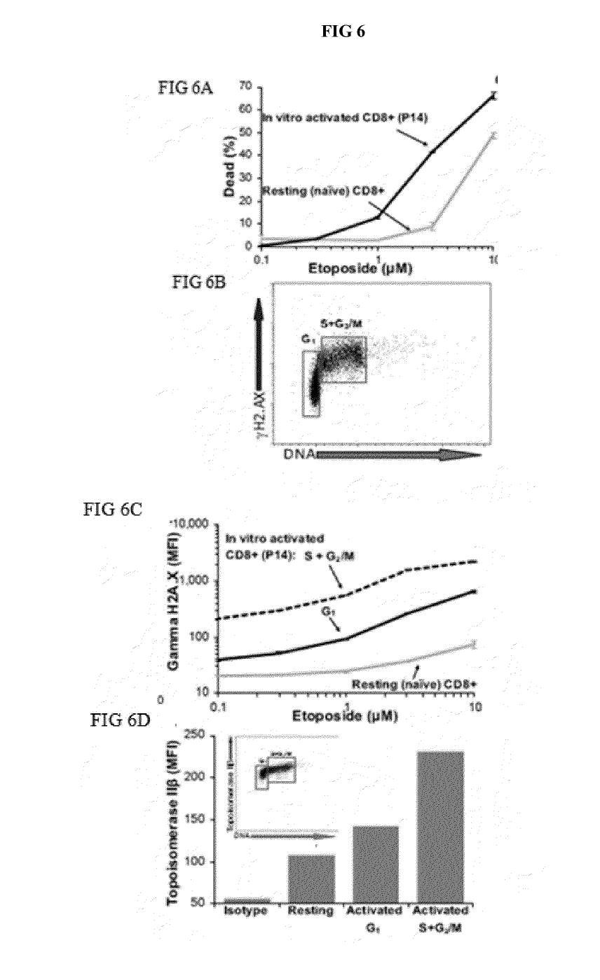

Mechanistically, Applicant has found that activated (as compared to resting) T cells display a substantial shift in their dose: response curve for etoposide mediated death (FIG. 6A). Two features potentially explain this differential sensitivity. First, they have increased baseline DNA damage (FIG. 5). Closer analysis reveals that highly activated T cell populations are bimodal, with cycling (S, G2/M) cells displaying even higher damage (FIG. 6B). This is likely due to replicative stress, and suggests that manipulating the G1/S checkpoint will be therapeutically useful (see below). Second, activated T cells in all phases of the cell cycle display a steeper dose:response relationship between etoposide exposure and DNA damage (FIG. 5C). FIG. 6D illustrates one potential mechanism for this increased sensitivity: activated T cells have higher levels of topoisomerase-II, the target molecule to which etoposide binds. Resistance to etoposide in tumor lines is highly correlated with decreased topoisomerase-II expression. FIG. 6 shows that activated T cells are more sensitive to etoposide-induced DNA damage/apoptosis induction and express increased levels of the target molecule, topoisomerase II. Resting (naïve) or activated CD8+ T cells (P14) were cultured with a titration of etoposide. Referring to FIG. 6A, cell death was assessed after overnight culture by 7-AAD/PS staining. Referring to FIG. 6B, DNA damage was measured by gamma-H2.AX staining, in conjunction with cell cycle analysis, after a four hour exposure to etoposide. Gamma-H2.AX intensity is plotted against etoposide dose for resting (G1) and activated T cells (G1 or S+G2/M). Referring to FIG. 6C, a representative dot plot of activated T cells is shown. Referring to FIG. 6D, topoisomerase II staining of resting and activated T cells is shown.

DNA damage triggers a well-studied series of events, including activation of ATM/ATR, p53, and downstream mechanism leading to DNA repair, senescence/cell cycle arrest, and/or cell death. P53 is widely considered to be a master integrator of cellular stress, promoting cell cycle arrest, senescence, DNA repair, and apoptosis in varying measures based on diverse inputs and contexts. Multiple proteins are known to regulate the strength and specificity of p53 signaling via phosphorylation, acetylation, ubiquitination, and other mechanisms. Specifically, MDM2 and MDM4 (or MDMX) are major regulators of p53 activity; both knockouts display p53-dependent embryonic lethality. They both bind to p53 and sequester it; decreasing transactivation and hastening its degradation in a complex, highly regulated fashion. Rationally designed small molecule inhibitors of both of these proteins have been developed, which ‘release’ p53. MDM2 inhibitors (the prototypical drug, called nutlin-3, referred to as simply ‘nutlin’ herein) have been tested in clinical trials as potentiators of cancer chemotherapy. Because nutlin enhances p53 function, it may also protect non-malignant cells (with non-mutant p53) from accumulating DNA damage in response to chemotherapy. Two MDM2 inhibitors are currently in clinical trials (RO5045337 and CGM097, see clinical trials.gov). MDM4 (and dual MDM2/4) inhibitors are in pre-clinical development. Acetylation of p53 promotes its transcriptional function, in part by destabilizing the p53-MDM2 interaction. P53 deacetylating proteins, including SIRT1, can have a significant negative impact on p53 function.

The DDR promotes DNA repair and survival by a variety of mechanisms, including cell cycle arrest. G1/S cell cycle arrest is promoted by p53 (largely via p21) and ATM/ATR (via Chk1 and other mediators). Concurrent with cell cycle arrest, repair mechanisms are engaged, involving Rad51 and other molecules. Multiple agents are in pre-clinical and clinical testing which interfere with the normal DDR in order to potentiate cancer chemotherapy. These agents include rationally designed, specific inhibitors of DNA-PK, CHK1/2, ATM/ATR, MDM2, SIRT1, CDK's, RAD51, and others.

Because activated T cells display an increased sensitivity to DNA damaging agents, it is believed that agents which potentiate the pro-apoptotic effects of p53 or inhibit DNA repair mechanisms would synergize with etoposide for the selective destruction of activated T cells. This synergy would produce more potent immunomodulatory effects and allow decreased doses of DNA damaging agents. Second, because activated T cells display a strong intrinsic DDR in vivo, without being limited by theory, it is believed that novel combinations which optimally exploit the pro-apoptotic potential of the DDR would allow antigen-specific immunomodulation without exogenous DNA damaging agents and with minimal or no off-target genotoxicity.

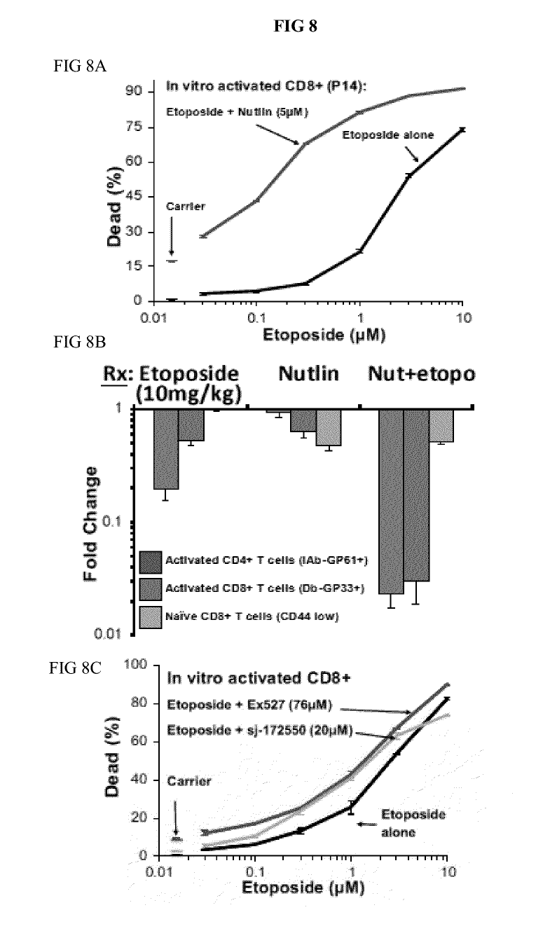

Applicant has conducted screening studies to begin testing these novel hypotheses and have identified strategies which are highly promising for further study. FIG. 8 illustrates that nutlin (an MDM2 inhibitor) dramatically shifts the etoposide:death, dose:response curve for activated T cells in vitro and potentiates etoposide immunomodulation in vivo (tested with a therapeutically suboptimal dose of etoposide). FIG. 6 shows that potentiators of p53 synergize with etoposide for killing of activated, but not resting, T cells. Referrring to FIG. 8A, in vitro activated T cells (P14) were cultured overnight with a titration of etoposide+/−5 uM nutlin and death was assessed. Referring to FIG. 8B, LCMV-infected animals were treated with low dose etoposide (10 mg/kg, instead of 50 mg/kg)+/−nutlin (50 mg/kg) on day 5 of infection. Antigen specific T cells were enumerated in the spleen by MHC (class I or Class II) tetramer staining on day 8 (the peak of the response). Referring to FIG. 8C, in vitro activated T cells (P14) were cultured with a titration of etoposide+/−the MDM4 inhibitor, Sj-172550, or the SIRT1 inhibitor, Ex527 and death was assessed after 14 hours. Similarly, Sj-172550, an MDM4 inhibitor, and Ex527, a SIRT1 inhibitor (2 targets which suppress p53 function), enhance etoposide killing of activated T cells in vitro. Though these initial studies reveal only modest shifts, it is expected that combination with drugs such as nutlin may reveal substantial synergies.

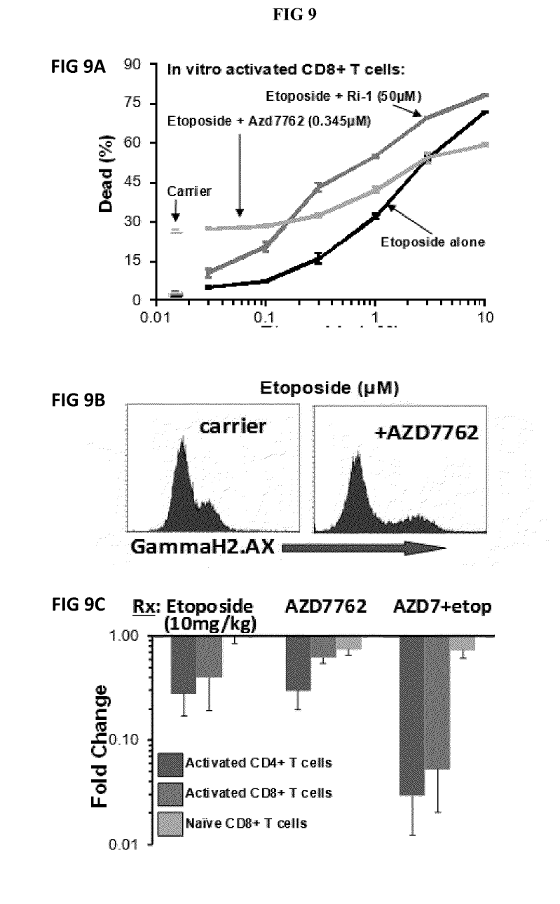

FIG. 9 illustrates that a Rad51 inhibitor and a CHK1/2 inhibitor (AZD7762) kill activated T cells in vitro and AZD7762 strongly synergizes with etoposide in vivo for the selective ablation of activated T cells. FIG. 9 shows that inhibitors of the DDR synergize with etoposide for killing of activated, but not resting T cells. Referring to FIG. 9A, in vitro activated T cells were incubated (overnight) with a titration of etoposide+/−a RAD51-specific inhibitor (Ri-1) or a CHK1/2 inhibitor (AZD7762) and death was assessed the next morning. Referring to FIG. 9B, gammaH2.AX staining of activated T cells after overnight culture+/−AZD7762 is shown. Referring to FIG. 9C, LCMV-infected animals were treated with low dose etoposide (10 mg/kg)+/−AZD7762 (25 mg/kg) on day 5 of infection. Antigen-specific T cells were assessed in the spleen on day 8 by MHC tetramer staining. Culture with AZD7762 (alone) leads to accumulation of spontaneous DNA damage in activated T cells which are in S+G2/M (but not those in G1), suggesting a mechanism for its selectivity.

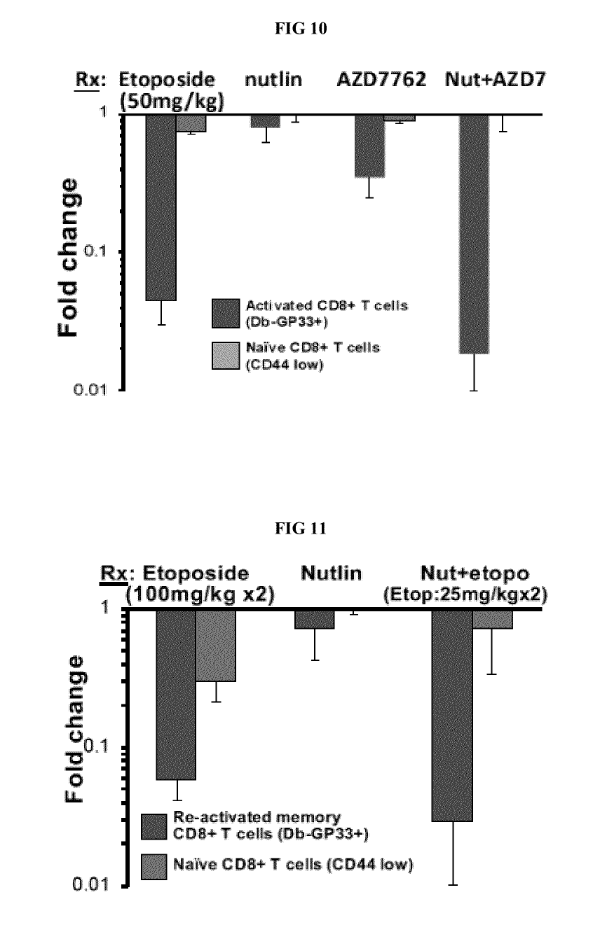

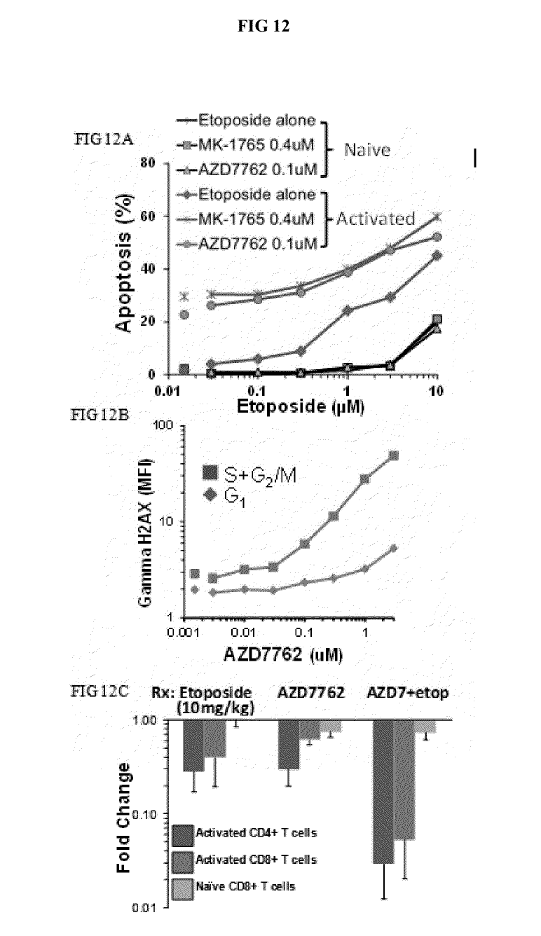

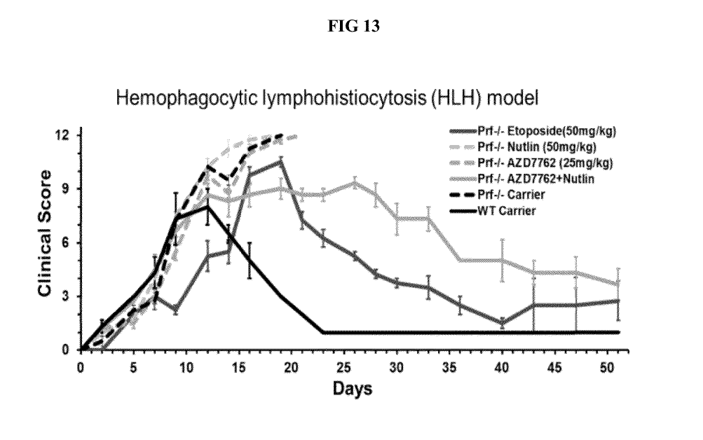

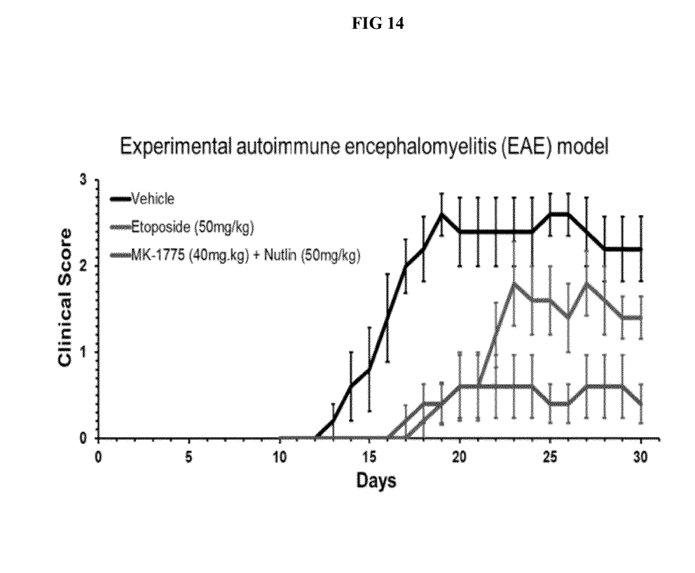

Finally, FIG. 10 demonstrates that inhibition of MDM2 and CHK1/2 gives highly efficient and selective ablation of activated T cells in vivo (nearly 100-fold loss), on par with full dose etoposide, but without DNA damaging agents. FIG. 10 shows potentiators of p53 and inhibitors of the DDR can synergistically eliminate activated T cells in vivo. LCMV-infected animals were treated with either standard dose etoposide (50 mg/kg, day 5), nutlin (50 mg/kg, ×4 on days 5 and 6), AZD7762 (25 mg/kg×2 on days 5 and 6), or Nutlin+AZD7762. Antigen specific CD8+ T cells were enumerated in the spleen on day 8 by MHC tetramer staining. These studies have provided a strong rationale for exploring the full therapeutic and adverse effect profiles of agents which target select aspects of the DDR+/−etoposide, as more beneficial immunotherapeutic approaches to avoid genotoxicity issues associated with etoposide.

Additional preliminary studies (not shown) demonstrate a clear timing and dose relationship for etoposide therapy; days 4-6 are optimal in the context of LCMV infection; 50-100 mg/kg is optimal. Pilot studies demonstrate that selective depletion of activated memory cells in WT mice is also feasible (FIG. 11). FIG. 11 shows that etoposide and p53 potentiators can synergistically ablate reactivated memory cells in vivo. WT mice were infected with LCMV. 1-2 months later, animals were injected with liposomal GP33 peptide (formulated similar to that taught in Zaks, K., et al., Efficient immunization and cross-priming by vaccine adjuvants containing TLR3 or TLR9 agonists complexed to cationic liposomes. J Immunol, 2006. 176(12): p. 7335-45). Two and 3 days later, animals were treated with carrier, etoposide (100 mg/kg/dose×2 doses), nutlin (50 mg/kg every 12 hours, for 4 doses), or both drugs (but with etoposide reduced to 25 mg/kg/dose). These studies were designed to be most relevant to clinical autoimmunity, where patients typically present with established disease, and pathologic memory T cell responses. In these studies, pre-established memory T cells are activated with a synthetic vaccine (instead of viral infection) and ablated with either etoposide, or the combination of lower dose etoposide+nutlin. It was found that nutlin and etoposide synergized impressively for the depletion of memory T cells which were reactivated in vivo with a synthetic vaccine (instead of viral infection). Published pharmacokinetic studies indicate that conventional bolus (i.p.) dosing of etoposide results in high (>30 uM) concentrations and rapidly falling levels in blood and tissues (FIG. 10). FIG. 6 illustrates that lower concentrations of etoposide (1-3 uM) are most selective for activated T cells over quiescent ones.

In summary, compelling preliminary studies indicate that novel combinations exploiting the DDR may enhance the action of etoposide and/or substitute entirely for an exogenous DNA-damaging agent, with regard to depletion of activated T cells and/or B cells in vivo. These combinations may display significant efficacy for alleviating murine HLH and other immunopathological conditions as described herein.

Example Dosing Regimen for HLH

A patient diagnosed with HLH is administered once daily a predetermined dose of nutlin and AZD daily upon first presentation. In mice this regimen was given on day 5 and 6 after LCMV infection, at the onset of extreme inflammation. The dosage used is in mice is 50 mg/kg nutlin, 25 mg/kg AZD

Example Dosing Regimen for EAE

A patient diagnosed with EAE is administered once daily nutlin and AZD upon symptom onset. In mice it was given on day 5 and 9 after MOG peptide vaccination. The dosage used is in mice is nutlin 50 mg/kg, MK 40 mg/kg.

### REFERENCES

1. Jordan, M. B., et al., An animal model of hemophagocytic lymphohistiocytosis (HLH): CD8+ T cells and interferon gamma are essential for the disorder. Blood, 2004. 104(3): p. 735-43.

2. Trottestam, H., et al., Chemoimmunotherapy for hemophagocytic lymphohistiocytosis: long-term results of the HLH-94 treatment protocol. Blood, 2011. 118(17): p. 4577-84.

3. Jordan, M. B., et al., How I treat hemophagocytic lymphohistiocytosis. Blood, 2011. 118(15): p. 4041-52.

4. Ambruso, D. R., et al., Successful treatment of lymphohistiocytic reticulosis with phagocytosis with epipodophyllotoxin VP 16-213. Cancer, 1980. 45(10): p. 2516-20.

5. Trottestam, H., et al., Chemoimmunotherapy for hemophagocytic lymphohistiocytosis: long-term results of the HLH-94 treatment protocol. Blood, 2011. 118(17): p. 4577-84.

6. Hande, K. R., Etoposide: four decades of development of a topoisomerase II inhibitor. Eur J Cancer, 1998. 34(10): p. 1514-21.

7. Clarke, A. R., et al., Thymocyte apoptosis induced by p53-dependent and independent pathways. Nature, 1993. 362(6423): p. 849-52.

8. Darzynkiewicz, Z., et al., Cytometry of DNA replication and RNA synthesis: Historical perspective and recent advances based on “click chemistry”. Cytometry A, 2011. 79(5): p. 328-37.

9. Asano, K., et al., Masking of phosphatidylserine inhibits apoptotic cell engulfment and induces autoantibody production in mice. J Exp Med, 2004. 200(4): p. 459-67.

10. Takano, H., et al., DNA topoisomerase-targeting antitumor agents and drug resistance. Anticancer Drugs, 1992. 3(4): p. 323-30.

11. Al-Ejeh, F., et al., Harnessing the complexity of DNA-damage response pathways to improve cancer treatment outcomes. Oncogene, 2010. 29(46): p. 6085-98.

12. de Rozieres, S., et al., The loss of mdm2 induces p53-mediated apoptosis. Oncogene, 2000. 19(13): p. 1691-7.

13. Parant, J., et al., Rescue of embryonic lethality in Mdm4-null mice by loss of Trp53 suggests a nonoverlapping pathway with MDM2 to regulate p53. Nat Genet, 2001. 29(1): p. 92-5.

14. Shen, H. and C. G. Maki, Pharmacologic activation of p53 by small-molecule MDM2 antagonists. Curr Pharm Des, 2011. 17(6): p. 560-8.

15. van Leeuwen, I. M., et al., An evaluation of small-molecule p53 activators as chemoprotectants ameliorating adverse effects of anticancer drugs in normal cells. Cell Cycle, 2012. 11(9): p. 1851-61.

16. Pei, D., Y. Zhang, and J. Zheng, Regulation of p53: a collaboration between Mdm2 and Mdmx. Oncotarget, 2012. 3(3): p. 228-35.

17. van Leeuwen, I. and S. Lain, Sirtuins and p53. Adv Cancer Res, 2009. 102: p. 171-95.

18. Srinivasan, A. and B. Gold, Small-molecule inhibitors of DNA damage-repair pathways: an approach to overcome tumor resistance to alkylating anticancer drugs. Future Med Chem, 2012. 4(9): p. 1093-111.

19. Luo, Y. and J. D. Leverson, New opportunities in chemosensitization and radiosensitization: modulating the DNA-damage response. Expert Rev Anticancer Ther, 2005. 5(2): p. 333-42.

20. Johnson, N. and G. I. Shapiro, Cyclin-dependent kinases (cdks) and the DNA damage response: rationale for cdk inhibitor-chemotherapy combinations as an anticancer strategy for solid tumors. Expert Opin Ther Targets, 2010. 14(11): p. 1199-212.

21. Zaks, K., et al., Efficient immunization and cross-priming by vaccine adjuvants containing TLR3 or TLR9 agonists complexed to cationic liposomes. J Immunol, 2006. 176(12): p. 7335-45.

22. Kurtulus, S. and D. Hildeman, Assessment of CD4(+) and CD8 (+) T cell responses using MHC class I and II tetramers. Methods Mol Biol, 2013. 979: p. 71-9.

23. Aisner, J., et al., A phase I trial of continuous infusion VP16-213 (etoposide). Cancer Chemother Pharmacol, 1982. 7(2-3): p. 157-60.

24. Blasius, M., et al., A phospho-proteomic screen identifies substrates of the checkpoint kinase Chk1. Genome Biol, 2011. 12(8): p. R78.

25. Inomata, M., et al., Regulation of Toll-like receptor signaling by NDP52-mediated selective autophagy is normally inactivated by A20. Cell Mol Life Sci, 2012. 69(6): p. 963-79.

26. Vanlangenakker, N., et al., TNF-induced necroptosis in L929 cells is tightly regulated by multiple TNFR1 complex I and II members. Cell Death Dis, 2011. 2: p. e230.

27. Baines, C. P., et al., Loss of cyclophilin D reveals a critical role for mitochondrial permeability transition in cell death. Nature, 2005. 434(7033): p. 658-62.

28. Kurtulus, S., et al., Bc1-2 allows effector and memory CD8+ T cells to tolerate higher expression of Bim. J Immunol, 2011. 186(10): p. 5729-37.

29. Kracikova, M., et al., A threshold mechanism mediates p53 cell fate decision between growth arrest and apoptosis. Cell Death Differ, 2013. 20(4): p. 576-88.

30. Takeda, K., et al., Induction of tumor-specific T cell immunity by anti-DR5 antibody therapy. J Exp Med, 2004. 199(4): p. 437-48.

31. Merino, D., et al., TRAIL in cancer therapy: present and future challenges. Expert Opin Ther Targets, 2007. 11(10): p. 1299-314.

32. Spitzer, D., et al., A genetically encoded multifunctional TRAIL trimer facilitates cell-specific targeting and tumor cell killing. Mol Cancer Ther, 2010. 9(7): p. 2142-51.

33. Bittker, J. A., et al., Discovery of Inhibitors of Anti-Apoptotic Protein A1, in Probe Reports from the NIH Molecular Libraries Program. 2010: Bethesda (Md.).

34. Wojciechowski, S., et al., Bim/Bc1-2 balance is critical for maintaining naive and memory T cell homeostasis. J Exp Med, 2007. 204(7): p. 1665-75.

35. Hammarlund, E., et al., Duration of antiviral immunity after smallpox vaccination. Nat Med, 2003. 9(9): p. 1131-7.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. The articles “a”, “an”, and “the” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses variations, combinations, and permutations in which one or more limitations, elements, clauses, descriptive terms, etc., from one or more of the claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth herein. It should also be understood that any embodiment of the invention, e.g., any embodiment found within the prior art, can be explicitly excluded from the claims, regardless of whether the specific exclusion is recited in the specification.

It should also be understood that, unless clearly indicated to the contrary, in any methods claimed herein that include more than one act, the order of the acts of the method is not necessarily limited to the order in which the acts of the method are recited, but the invention includes embodiments in which the order is so limited. Furthermore, where the claims recite a composition, the invention encompasses methods of using the composition and methods of making the composition. Where the claims recite a composition, it should be understood that the invention encompasses methods of using the composition and methods of making the composition.

All percentages and ratios are calculated by weight unless otherwise indicated. All percentages and ratios are calculated based on the total composition unless otherwise indicated.

It should be understood that every maximum numerical limitation that may be given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

To the extent dimensions and values are disclosed herein, such are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A composition comprising an agent selected from a p53 potentiating agent, a DNA-damaging agent, a DNA repair inhibitor/cell cycle checkpoint inhibitor, and combinations thereof, and a pharmaceutically acceptable carrier.

2. The composition according to claim 1, wherein said p53 potentiating agent is selected from an MDM2 inhibitor, an MDM4 inhibitor, a dual MDM2/MDM4 inhibitor, a SIRT 1 inhibitor, and combinations thereof.

3. The composition of claim 1, wherein said p53 potentiating agent is a MDM2 inhibitor.

4. The composition of claim 1, wherein said p53 potentiating agent is a nutlin compound.

5. The composition of claim 1, wherein said p53 potentiating agent is selected from nutlin 1, nutlin 2, nutlin 3, or combinations thereof.

6. The composition according to claim 1, wherein said DNA damaging agent is selected from a topoisomerase type I inhibitor, a topoisomerase type II inhibitor, an alkylating agent, an antimetabolite, a cytotoxic antibiotic, a purine analogue, a dihydrofolate reductase inhibitor, and combinations thereof

7. The composition of claim 1, wherein said DNA damaging agent is a topoisomerase type II inhibitor

8. The composition of claim 1, wherein said DNA damaging agent is a topoisomerase type II inhibitor selected from etoposide, teniposide, doxorubicin, daunorubicin, mitoxantrone, amsacrine, ellipticines, aurintricarboxylic acid, HU-331, and combinations thereof.

9. The composition of claim 1, wherein said DNA damaging agent is etoposide.

10. The composition according to claim 1, wherein said DNA repair inhibitor/cell cycle checkpoint inhibitor is selected from a CHK1/2 Inhibitor, a Rad51 Inhibitor, a Wee 1 inhibitor, an ATR inhibitor, and combinations thereof.

11. The composition of claim 1, wherein said DNA repair inhibitor/cell cycle checkpoint inhibitor is a CHK 1/2 inhibitor.

12. The composition of claim 1, wherein said DNA repair inhibitor/cell cycle checkpoint inhibitor is AZD7762.

13. The composition of claim 1, wherein said DNA repair inhibitor/cell cycle checkpoint inhibitor is an ATR inhibitor.

14. The composition of claim 1, wherein said composition comprises a p53 potentiating agent, a DNA damaging agent, a DNA repair inhibitor/cell cycle checkpoint inhibitor, and a pharmaceutically acceptable carrier.

15. The composition of claim 1, wherein said composition comprises a p53 potentiating agent, a DNA-damaging agent, and a pharmaceutically acceptable carrier.

16. The composition of claim 1, wherein said composition comprises a p53 potentiating agent, a DNA repair inhibitor/cell cycle checkpoint inhibitor; and a pharmaceutically acceptable carrier.

17. The composition of claim 1 comprising a DNA repair inhibitor/cell cycle checkpoint inhibitor, and a pharmaceutically acceptable carrier

18. The composition of claim 1 comprising a CHK 1/2 inhibitor, a Wee1 inhibitor, and a pharmaceutically acceptable carrier.

19. The composition of claim 17, further comprising etoposide

20. The composition of claim 1 comprising a CHK 1/2 inhibitor, a wee1 inhibitor, a MDM2 inhibitor.

21. The composition of claim 19, further comprising etoposide.

22. A composition comprising a p53 potentiating agent and a DNA repair inhibitor/cell cycle checkpoint inhibitor, wherein said composition is substantially free of etoposide.

23. The composition of claim 22, wherein said DNA repair inhibitor/cell cycle checkpoint inhibitor is selected from an agent that inhibits CHK1/2 or Wee1 or a combination thereof, and wherein said p53 potentiating agent comprises an inhibitor of MDM2.

24. The composition of claim 23, wherein the composition is substantially free of etoposide.

25. A composition comprising a chemotherapeutic agent and a combination comprising a p53 potentiating agent and a DNA repair inhibitor/cell cycle checkpoint inhibitor.

26. The composition of claim 25wherein said p53 potentiating agent comprises an inhibitor of MDM2.

27. A method of treating a condition involving activated T cells and/or activated B cells, comprising the step of administering a composition according to claim 1.

28. The method of claim 28 wherein said condition is an immunological condition.

29. The method of claim 28 wherein said immunological condition is selected from allergies, autoimmune conditions, allo-immune conditions, and other pathological immune reactivities

30. The method of claim 28 wherein said immunological condition is selected from hemophagocytic lympohohistiocytosis, graft versus host disease, EAE, lupus nephritis, multiple sclerosis, rheumatoid arthritis, autoimmune encephalitis, allogenic graft rejection, transfusion reactions, allergies, and anti-drug immune responses

31. The method of claim 28 wherein said immunological condition is hemophagocytic lympohohistiocytosis (HLH)

32. The method of claim 27wherein said method reduces activated T cells and/or B cells in vivo.

33. The method of claim 27 wherein said method selectively modulates immune function.

34. The method of claim 27 wherein said method selectively reduces the activity of or ablates activated T cells.

35. The method of claim 27 wherein said method induces selective tolerance to an agent activating an immune response of an individual.

36. The method of claim 27, comprising administering a composition comprising a p53 potentiating agent and a DNA repair inhibitor/cell cycle checkpoint inhibitor, wherein said composition is substantially free of etoposide.

37. The method of claim 36, wherein said DNA repair inhibitor/cell cycle checkpoint inhibitor is selected from an agent that inhibits CHK1/2 or Wee1, or ATR, or a combination thereof, and wherein said p53 potentiating agent comprises an inhibitor of MDM2.

38. The method of claim 36, wherein the composition is substantially free of etoposide.

39. The method of claim 27, comprising administering a composition comprising a chemotherapeutic agent and a combination comprising a p53 potentiating agent and a DNA repair inhibitor/cell cycle checkpoint inhibitor.

40. The method of claim 39 wherein said p53 potentiating agent comprises an inhibitor of MDM2.

41. A method of enhancing the effectiveness of etoposide, comprising the step of administering an agent selected from a p53 potentiating agent, a DNA repair inhibitor/cell cycle checkpoint inhibitor, or a combination thereof.
